(19) United States
(12) Patent Application Publication
(10) Pub. No.: US 2003/0124590 A1
(43) Pub. Date: Jul. 3, 2003

(54) Carbohydrate response element binding protein and uses thereof

(76) Inventor: Kosaku Uyeda, Dallas, TX (US)

Correspondence Address: BAKER & BOTTS, 30 ROCKEFELLER PLAZA, NEW YORK, NY, 10112

(21) Appl. No.: 10/272,206

(22) Filed: Oct. 16, 2002

Related U.S. Application Data
(60) Provisional application No. 60/329,834, filed on Oct. 16, 2001.

(51) Int. Cl. A61K 31/00; C12Q 1/68

(52) U.S. Cl. 435/6; 514/1

(57) ABSTRACT

The present invention relates to the field of transcriptional regulation. More specifically, it relates to a novel transcription factor, Carbohydrate Response Element Binding Protein (ChREBP). ChREBP is associated with carbohydrate metabolism and the conversion of dietary excess carbohydrate to body fat. The present invention relates to activation and inhibition of ChREBP transcriptional activity and uses thereof.

### SPECIFICATION

The present application claims priority to U.S. Provisional Application Serial No. 60/329,834, filed Oct. 16, 2001, which is incorporated herein by reference in its entirety.

The present invention involves subject matter developed under NIH Grant No. DKK 16194 and Veterans Administration Merit Review #97-119. Consequently, the United States Government may have certain rights herein.

### FIELD OF THE INVENTION

The present invention relates to the field of transcriptional regulation. More specifically, it relates to a novel transcription factor, Carbohydrate Response Element Binding Protein (ChREBP). ChREBP is associated with carbohydrate metabolism and the conversion of dietary excess carbohydrate to body fat. The present invention relates to activation and inhibition of ChREBP transcriptional activity and uses thereof.

### BACKGROUND OF THE INVENTION

A diet high in excess carbohydrate stimulates conversion of carbohydrate into storage fat in the mammalian liver. Accumulation of an abundance of simple carbohydrates, such as glucose, are efficiently stored as fat. Evolutionary pressures have favored the emergence of “thrifty genes” in humans to safeguard against periodic famine (Neel, J. N. (1999) Nutr. Rev. 57, S2-S9). However, the phenomenon of an abundance of food and food rich in carbohydrate, in conjunction with modem lifestyles, has contributed to health defects, such as a diabetes, obesity, heart disease etc.

The liver responds in two ways to the increase in carbohydrate intake after a meal: 1) acute activation of various rate-limiting glycolytic and lipogenic enzymes and 2) upregulation of gene expression of these enzymes. Lipogenesis is the formation of fats, such as triglycerides from simple carbohydrates, such as glucose. Examples of glycolytic and lipogenic enzymes include L-type pyruvate kinase (L-PK), acetyl CoA carboxylase, and fatty acid synthase. It is the transcriptional upregulation of these enzymes that promotes conversion and long term storage of carbohydrates to triglycerides.

Hormones, such as insulin and glucagon, play a critical role in regulating glucose metabolism and storage (reviewed in Granner and Pilkis (1990) J. Biol. Chem. 265, 10173-10176). Insulin, for example, induces expression of glucokinase, an enzyme in glycolysis.

L-type pyruvate kinase (L-PK), for example, plays a central role in glucose metabolism by regulating the flux of phosphoenolpyruvate and pyruvate. The accumulation of pyruvate promotes conversion to triglycerides with high glucose in circulation after a diet of high carbohydrate.

Glucose also modulates the transcription of many other genes involved in lipogenesis. Glucose stimulates L-PK gene transcription, independent of insulin, in cultured hepatocytes expressing active glucokinase (Doiron et al (1994) J. Biol. Chem. 269, 10213-10216). Starvation and diabetes in rats produce reduced L-PK mRNA levels and enzymatic activity, which are restored to normal levels and activity with refeeding and insulin (Noguchi et al. (1985) J. Biol. Chem. 260, 14393-14397; Vaulont et al. (1986) J. Biol. Chem. 261, 7621-7625). However, the mechanism by which excess glucose activates transcription of glycolytic and lipogenic enzymes remains unclear.

Transcription of such metabolic enzymes, for example, L-PK, fatty acid synthase, acetyl CoA carboxylase and spot-14 protein (S14), is mediated in a glucose-responsive manner through regulatory elements called the glucose response element (GRE) or carbohydrate response element (CRE) located in the promoter regions upstream from the genes (Granner and Pilkis (1990) J. Biol. Chem. 265, 10173-10176; Liu et al. (1993) J. Biol. Chem. 268, 12787-12795; Bergot et al. (1992) Nucleic Acids Res. 20, 1871-1877; Cuif et al. (1993) J. Biol. Chem. 268, 13769-13772; Thompson et al. (1991) J. Biol. Chem. 268, 8679-8682). In L-PK, the glucose or carbohydrate response element is located in the region −183 to −96 base pairs upstream from the cap site of the L-PK gene (Shih et al. (1995) J. Biol. Chem. 270, 21991-21997). Although a substantial effort has been made to study the promoter regions of these genes, a glucose-responsive mechanism of action has yet to emerge (Kaytor et al. (1997) J. Biol. Chem. 272, 7525-7531; Koo and Towle (2000) J. Biol. Chem. 275, 5200-5207; Yamada et al. (1999) Biochem and Biophys Res Comm 257, 44-49).

Transcription factors are proteins that bind to regulatory elements in the promoter regions of genes and play a critical role in gene regulation and protein expression contributing to homeostasis, development, cellular growth and differentiation. They may serve to either activate or repress expression of the gene. Transcription factors generally can be categorized into four major groups according to the motif in their DNA-binding domains which include (1) the helix-turn-helix group, (2) the zinc finger group, (3) the leucine zipper group, and (4) the helix-loop-helix/leucine zipper group.

ChREBP is a novel transcription factor that binds in a glucose responsive manner to GRE (glucose response element) or CRE (carbohydrate response element) in the promoter regions of genes encoding glycolytic or lipogenic enzymes. ChREBP is a member of the basic helix-loop-helix, leucine zipper (bHLH/Zip) family of transcription factors, has a Mr=94,600 and contains several potentially functional domains, including a nuclear localization signal (NLS), a proline-rich stretch (PRO), a basic helix-loop-helix, leucine-zipper (bHLH/ZIP), and a leucine zipper-like (ZIP-like) domain (FIG. 5) (Yamashita et al. (2001) Proc. Natl. Acad. Sci. U.S.A. 98, 9116-9121). It contains three consensus phosphorylation sites for cAMP-dependent protein kinase (PKA). It is expressed in the liver, where lipogenesis occurs, and may have a critical role in regulating long term storage of excess carbohydrates to fats.

Furthermore, cAMP may regulate ChREBP activity (FIG. 15). At least one of the three consensus phosphorylation sites is located within the basic region of the putative DNA-binding domain. When circulating carbohydrate levels are low, elevated glucagon levels promote increased cAMP production, which activates PKA and may result in phosphorylation of ChREBP and inactivation of DNA binding and transcriptional activity. Conversely, excess carbohydrates would lower cAMP levels and also lead to the activation of a protein phosphatase that hydrolyzes the phosphoryl group from inactive ChREBP. Dephosphorylation of inactivated ChREBP would restore DNA-binding activity and induce transcription of genes required for carbohydrate metabolism and conversion to fat.

The present invention relates to the use of a novel transcription factor to regulate activity of enzymes and hormones involved in carbohydrate metabolism. The present invention provides for methods for inhibiting the process of lipogenesis. Further, the present invention also provides methods for identifying agents to inhibit the activity of enzymes involved in carbohydrate metabolism.

### SUMMARY OF THE INVENTION

The present invention relates to the discovery of a novel transcription factor, called ChREBP (Carbohydrate Response Element Binding Protein) which functions in glucose responsive regulation of glycolytic and lipogenic enzyme gene expression. The present invention also relates to the discovery of regulation of ChREBP through phosphorylation. ChREBP contains 3 PKA consensus phosphorylation sites which modulate nuclear transport and DNA binding activity. Dephosphorylation of these sites mediate import of ChREBP into the nucleus and DNA binding to promoter regions.

In an embodiment, the present invention provides for a method of regulating expression of genes encoding glycolytic and lipogenic enzymes.

In another embodiment, the present invention provides for methods of inhibiting gene expression of genes encoding glycolytic and lipogenic enzymes.

In yet another embodiment, the present invention provides methods of inhibiting fat storage (storage of sugars as triglycerides).

In a further embodiment, the present invention provides methods for the treatment of obesity, diabetes and vascular diseases by inhibiting glycolytic and lipogenic enzyme gene expression.

The invention provides for treatment of obesity, diabetes or cardiac diseases in an individual by administering to an individual in need of treatment an agent that regulates ChREBP activity. The invention also provides methods of treatment of disorders obesity, diabetes, cardiac diseases by administering agents that antagonize (inhibit) ChREBP function.

Methods to identify ChREBP agonists and antagonists, are also provided by the invention.

### DESCRIPTION OF THE FIGURES

FIG. 1 is a gel-shift assay showing that a high carbohydrate diet induces ChREBP DNA-binding activity in rat livers.

FIGS. 2A-2B demonstrate that the specificity of ChREBP DNA binding in vitro correlates with promoter activity in vivo. FIG. 2A is a gel shift assay that shows the specificity of ChREBP binding to L-PK wild type and mutant ChRE oligonucleotides. FIG. 2B is a bar graph showing that ChREBP binding to L-PK wild type and mutant ChRE oligonucleotides corresponds to its ability to mediate transcription of a luciferase reporter gene.

FIG. 3A is a Northern blot showing the tissue distribution of ChREBP mRNA. FIG. 3B is a gel shift assay showing that ChREBP activity is only detected in liver nuclear extracts.

FIG. 4 is a bar graph demonstrating ChREBP activation of luciferase reporter gene expression from the LPK promoter in response to 10 mM lactate, 5.5 mM glucose or 27.5 mM glucose in vivo.

FIG. 5 shows a schematic of ChREBP protein motifs including a bipartite nuclear localization signal (NLS), a bHLH-ZIP motif and three consensus PKA phosphorylation sites (P1, P2, P3) including one (sequence RRIT) within the bHLH region.

FIG. 6 is bar graph showing results from a gel shift assay showing that DNA-binding activity of purified ChREBP can be regulated by phosphorylation in vitro.

FIG. 7 shows the effects of domain deletion of ChREBP on transcriptional activity of L-PK gene.

FIG. 8 is a bar graph showing the effects of PKA inhibitor, H-89, and PP2A inhibitor, cantharidic acid, on transcription activity of L-PK gene in ChREBP overexpressed hepatocytes.

FIG. 9 shows immunofluorescent micrographs of subcellular localization of GFP-fused ChREBP transfected in cultured hepatocytes grown under low or high glucose conditions.

FIG. 10 is a bar graph showing the effect of NLS domain deletion, H-89, and cantharidic acid on nuclear translocation of ChREBP.

FIG. 11A shows the putative PKA phosphorylation sites and substitutions introduced by site-directed mutagenesis. FIG. 11B is a bar graph that shows the effect of mutation of Ser¹⁹⁶ on subcellular localization of ChREBP.

FIG. 12 is a gel shift assay showing that intraperitoneal injection of db-cAMP into rats inhibits ChREBP binding in vivo.

FIG. 13 shows that the C-terminal fragment of ChREBP exhibits phosphorylation dependent binding activity. FIG. 13A is a graph showing that phosphate incorporation mediated by PKA correlates directly with DNA binding. FIG. 13B a graph showing rates of dephosphorylation of ChREBP by PP2A and the activation of DNA-binding activity of ChREBP in vitro.

FIGS. 14A and 14B are bar graphs showing the effect of ChREBP mutants on L-PK promoter driven reporter gene activity.

FIG. 15 is a schematic representation of regulatory mechanisms of ChREBP by glucose and cAMP in hepatocytes.

FIG. 16 demonstrates that ChREBP binds to GRE and ChRE of genes such as spot 14 protein (S 14), insulin (InsZ), fatty acid synthase (FAS), acetyl CoA carboxylase (ACC), and ATP citrate lyase (ACL). FIG. 16A is a bar graph quantifying the gel shift assay of FIG. 16B.

FIG. 17 is an alignment of rat, mouse and human ChREBP amino acid sequences.

FIG. 18 is a bar graph showing the effect of various fatty acids on glucose-induced ChREBP-mediated L-PK promoter driven reporter gene activity.

FIG. 19 is a bar graph showing the effects of fatty acids on AMPK activity in hepatocytes.

FIG. 20 is a bar graph showing the effects of AICAR on glucose-induced ChREBP-mediated L-PK promoter driven reporter gene activity.

FIG. 21A is a bar graph showing the relative phosphatase activity of liver extract from rats fed a high carbohydrate diet and a rat that was fasting. FIG. 21B is a bar graph showing the relative protein phosphatase activity of liver extract with various endogenous metabolites removed.

FIG. 22A is a bar graph showing DEAE cellulose chromatography fractions collected from rat liver cytosolic extract. FIG. 22B is a graph showing Mono Q chromatograph fractions collected from pooled DEAE fractions having Xu-5-P activated phosphatase activity. FIG. 22C is an immunoblot of fractions #22-25 collected from the Mono-Q column and blotted with antibodies to phosphatase PP2A subunits A, Bα, Bδ, and C.

FIG. 24 shows a bar graph of relative phosphastase activity of nuclear extract from fasted and high carbohydrate fed rats livers using P3 or P4 as the substrate with various endogenous metabolites removed.

FIG. 25A a bar graph showing DEAE cellulose chromatography fractions collected from rat liver nuclear extract. FIG. 25B is a graph showing Mono Q chromatograph fractions collected from pooled DEAE fractions having Xu-5-P activated phosphatase activity. FIG. 25C is an immunoblot of fractions #22-25 collected from the Mono-Q column and blotted with antibodies to phosphatase PP2A subunits A, Bα, Bδ, and C.

FIG. 26 is a bar graph showing the relative phosphatase activity of nuclear Xu-5-P activated phosphatase activity in the presence and absence of various phosphatase inhibitors using P3 (A) or P4(B) as a substrate.

FIG. 27 is a bar graph showing the kinetics of Xu-5-P formation, nuclear import and the L-PK transcriptional activity in primary hepatocytes.

### DETAILED DESCRIPTION OF THE INVENTION

This invention relates to the discovery of a novel transcription factor, ChREBP and uses thereof. This invention demonstrates that ChREBP binds to promoter elements of a number of genes encoding metabolic enzymes and other proteins, such as hormones involved in glycolysis and lipogenesis. These genes contain previously identified GRE (glucose response element) or ChRE (carbohydrate response element) in the promoter regions. However, the transcription factors which may bind to regulatory response elements and mediate transactivation of genes involved in glycolysis and lipogenesis have not been identified before the present invention.

The present invention provides for a newly isolated transcription factor, ChREBP, which binds to regulatory response elements and stimulates expression of genes operably linked to these genetic elements. Furthermore, the present invention describes structural features of ChREBP which define its mechanism of action.

The present invention is based in the observation that ChREBP activity is regulated by its phosphorylation state. Phosphorylated ChREBP protein remains inactive in the cytosol, whereas dephosphorylated ChREBP protein can be imported into the nucleus and bind to CRE or GRE of metabolic enzymes involved in glycolysis, gluconeogenesis and lipogenesis.

The term ChREBP as used herein includes naturally occurring mammalian ChREBP and also ChREBP that has been modified. Naturally occurring ChREBP is a protein having a molecular weight of 100 kD. The gene encoding ChREBP and the amino acid sequence of the encoded protein are known in the art as disclosed in deLuis et al (deLuis et al. (2000) Eur J Hum Genet 8, 215-222) and as Accession No. AF156604 (Genbank). Further, the term ChREBP as defined herein may include other modifications such as insertions, deletions and substitutions provided that ChREBP retains functions such as the functions of nuclear import and DNA binding.

The term DNA molecule as used herein includes molecules that encode proteins, including enzymes and hormones, involved in glycolysis or lipogenesis. Further, the term DNA molecule includes genes, their structural elements, their coding elements, their regulatory elements, introns, exons, and other sequences. In a preferred embodiment, the DNA molecule encodes a hormone or enzyme involved in glucose metabolism and lipogenesis. These proteins include L-type pyruvate kinase, fatty acid synthase, acetyl CoA carboxylase, and ATP citrate lyase. In a most preferred embodiment, the DNA molecule encodes L-type pyruvate kinase.

The term “glucose responsive” as used throughout the specification refers to the mechanism of ChREBP transactivation. Excess circulating glucose in the bloodstream stimulates the transcription of a number of DNA molecules encoding lipogenic and glycolytic proteins. Expression of these enzymes or hormones is upregulated in response to the increased amount of glucose.

ChREBP comprises at least one phosphorylation site, most preferably three consensus phosphorylation sites. At least two sites appear to regulate ChREBP import into the nucleus and DNA binding to response elements, ChRE and GRE. Phosphorylation of Serine 196 prevented import into the nucleus and phosphorylation of Threonine 666 impaired DNA binding and transactivation ability as shown in Example 10.

Dephosphorylated ChREBP can increase the transcription of DNA molecules encoding proteins, such as enzymes or hormones involved in lipogeneis or glucose metabolism by binding to the regulatory response elements in the promoter regions. Dephosphorylated ChREBP can also increase transcription by stimulating nuclear import and activation of DNA binding activity. Alternatively, phosphorylated ChREBP can decrease or substantially inhibit the transcription of the DNA molecule by inhibition of nuclear import and inhibition of DNA binding.

One embodiment of the invention provides a method for modulating expression of a DNA molecule encoding proteins involved in lipogenesis or glucose metabolism, that includes modulating the phosphorylation of the ChREBP that binds to response elements found in promoter regions of the DNA molecule. Preferably, the response elements are GRE or ChRE found in promoter regions of DNA molecules that encode glycolytic or lipogenic enzymes or hormones.

The phosphorylation state of ChREBP can be regulated by modulating the activity of a phosphatase that dephosphorylates ChREBP or the activity of a kinase that phosphorylates ChREBP. Modulation may include inhibition or stimulation of phosphatase or kinase activity. Other methods of dephosphorylating or phosphorylating ChREBP may also be used for modulating the phosphorylation state of ChREBP and are within the scope of the invention.

A variety of phosphatases and kinases are known in the art and can modulate the phosphorylation of ChREBP in the present invention. For example, the phosphatase may be a serine/threonine phosphatase. Phosphatases include, without limitation, PP1, PP2A, PP2C, PPIV, and PPV. In a preferred embodiment, the phosphatase is a PP2A phosphatase. Kinases may include cAMP activated protein kinase (PKA) and AMP activated protein kinase (AMPK). The data in the Examples section demonstrate the effect of inhibiting a kinase, PKA, and a phosphatase, PP2A on the activity of ChREBP.

In a preferred embodiment of the invention, ChREBP remains in the phosphorylated state and cannot translocate into the nucleus or bind to a carbohydrate responsive element. Phosphorylated ChREBP can decrease or inhibit the transcription of DNA molecules encoding proteins, such as enzymes or hormones involved in lipogenesis or glycolysis. Preferably, the reduced expression of some glycolytic and lipogenic proteins prevents the conversion of excess simple carbohydrates, such as glucose, to triglycerides.

The phosphorylation of ChREBP may be modulated by using a phosphatase that is capable of dephosphorylating ChREBP. Inhibition of phosphatase activity allows ChREBP to remain in a phosphorylated state, preventing translocation into the nucleus or binding to carbohydrate response element. The phosphatase inhibitor may also inhibit more than one type of phosphatase. In a preferred embodiment, the phosphatase inhibitor is a PP2A phosphatase inhibitor. Phosphatase inhibitors that can be used in the present invention may include, without limitation, okadaic acid (OA) and Calyculin A (CyA). For in vivo uses, the phosphatase inhibitor may be administered systemically or locally. Preferably, the phosphatase inhibitor is administered locally.

In a further embodiment, the invention provides for a method of inhibiting glucose conversion to triglycerides by modulating the phosphorylation of ChREBP to inhibit ChREBP binding to GRE or ChRE from a promoter region of a DNA molecule encoding a protein, such as an enzyme or hormone involved in lipogenesis or glucose metabolism.

In another embodiment, the invention provides for a method of identifying an agent which modulates phosphorylation of ChREBP that is involved in regulation of a gene encoding a hormone or enzyme involved in lipogenesis or glucose metabolism. The method includes (1) incubating the candidate agent with cellular proteins including ChREBP under conditions to allow the components to interact and (2) determining the effect on ChREBP in nuclear import, DNA binding and/or reporter gene expression. In a preferred embodiment, the invention provides for a method of identifying an agent which phosphorylates ChREBP to suppress expression of glycolytic and lipogenic genes.

Since ChREBP import appears to be regulated by the phosphorylation state of residue S¹⁹⁶, the invention provides for method of modulating phosphorylation of ChREBP using phosphatase and kinase inhibitors as described above.

Since ChREBP binding to DNA appears to be regulated by the phosphorylation state of residue T⁶⁶⁶, the invention provides for methods of modulating phosphorylation of ChREBP using phosphatase and kinase inhibitors as described above.

In alternative embodiment, the invention provides for a method of identifying an agent which binds to and/or blocks ChREBP binding at or near the response element. The method comprises incubating the candidate agent and the response element of the DNA molecule of interest and ChREBP under conditions which allow them to interact and measuring the effect on transcription using a functional assay.

In another embodiment, the invention provides for a method of identifying an agent which regulates ChREBP nuclear import through regulation of the nuclear localization signal (NLS). ChREBP transcriptional activity is dependent on localization to the nucleus. The method includes (1) incubating the candidate agent with ChREBP under conditions to allow the agent to interact with the NLS of ChREBP and (2) determining the effect on ChREBP in nuclear import using functional assays such as those described in the Examples. In a preferred embodiment, the invention provides for the identification of candidate agents which inhibit nuclear import of ChREBP.

Candidate agents that may be tested by the assays of the present invention include chemicals, proteins, peptides, non-peptide small molecules, biological agents and any other source of candidate agents having potential ChREBP regulating activity. The agents may be naturally occurring or synthetic, and may be a single substance or a mixture. Screening may be performed in high throughput format using combinatorial libraries, expression libraries and the like. Functional assays may include DNA bindings assays, reporter gene activity assays and fluorescent nuclear import assays as described in the Examples. Transcriptional activity may be measured by, but not limited to reporter genes such as CAT, luciferase, GFP, and β-gal.

Incubation of the test agents may include conditions which allow for contact between the ChREBP and the test agent in solution, solid phase, or in a cell. The test agents may be a combinatorial library for screening many agents.

### EXAMPLES

These examples further describe and demonstrate embodiments within the scope of the present invention. The examples are given solely for the purpose of illustration and are not to be construed as limitations of the present invention, as many variations thereof are possible without departing from the spirit and scope of the invention.

### Example 1

### Purification of ChREBP

A. Materials and Methods

Preparation of Nuclear Extracts. Rats were starved for 48 h and then fed 24 h with either the NIH high sucrose diet lab chow (20% casein, 60.2% sucrose, 15% cellulose, 2.25% minerals, and 2.25% vitamins) (Casazza et al (1986) Biochem J 236, 635-641), or a high fat diet without starch (31% casein, 30.5% cellulose, 7% minerals, 1.5% vitamins, 1.5% corn oil, 1.5% peanut oil, and 27% lard) (Francone et al. (1992) Am J Physiol 263, E615-E623). Liver nuclear extracts were prepared according to Hattori et al. (Hattori et al. (1990) DNA Cell Biol 9, 777-781) with minor modifications (Hasegawa et al. (1999) J Biol Chem 274, 1100-1107). Fresh rat livers (˜10 g) were homogenized with a Potter-Elvehjem homogenizer in extraction buffer (10 mM Hepes (pH 7.9), 10 mM KCl, 0.1 mM EDTA, 0.74 mM spermidine, 1 mM DTT, 0.5 mM PMSF) containing 0.3 M sucrose. The homogenate was mixed with 2 volumes of cushion buffer (extraction buffer containing 2.2 M sucrose), layered on top of cushion buffer, and centrifuged at 75,000×g for 30 min at 4° C. Pelleted nuclei were resuspended in 5 volumes of nuclear lysis buffer (10% glycerol, 10 mM Hepes (pH 7.9), 420 mM NaCl, 0.1 mM EDTA, 3 mM MgCl₂, 5 mM DTT, 0.5 mM PMSF, 0.5 μg/ml pepstatin A, 1 mM benzamidine, and 0.2 μg/ml leupeptin) and homogenized with 10 strokes of a hand-held Dounce homogenizer. The nuclear extracts were centrifuged at 27,000×g for 25 min at 4° C., and polyethylene glycol (PEG; Mr=8,000) was added to the supernatant solution to 25% saturation. Following centrifugation at 27,000×g for 10 min, the pellet was dissolved in a buffer containing 20% glycerol, 20 mM Hepes (pH 7.9), 100 mM KCl, 0.2 mM EDTA, 5 mM DTT, and 0.5 mM PMSF and cleared following centrifugation at 27,000×g for 5 min at 4° C. Protein concentration was determined using the Bradford method (Bradford (1976) Anal Biochem 72, 248-254). The addition of the PEG during extract preparation stabilized ChREBP and other transcription factors to allow for detection by gel shift. The PEG precipitated proteins can be assayed immediately without a lengthy dialysis.

Biochemical purification. Crude nuclear extract was prepared from the livers of 800 male Sprague-Dawley rats refed a high carbohydrate diet for 24 h following starvation for 48 h. Livers were homogenized in 2 volumes of buffer (300 mM sucrose, 10 mM Hepes (pH 7.9), 10 mM KCl, 0.1 mM EDTA, 0.74 mM spermidine, 1 mM DTT, 0.5 mM PMSF). The homogenate was mixed with 2 volumes of buffer containing 2.2 M sucrose, layered on top of buffer containing 2.2 M sucrose, and centrifuged at 75000×g for 45 min at 4° C. Pelleted nuclei were homogenized in 2.5 volumes of lysis buffer (10% glycerol, 10 mM Hepes (pH 7.9), 10 mM KCl, 0.1 mM EDTA, 3 mM MgCl₂, 5 mM DTT, 0.5 mM PMSF, 0.5 μg/ml pepstatin A, 1 mM benzamidine, 0.2 μg/ml leupeptin). Following addition of 0.1 volumes of 4.2 M NaCl, the homogenate was gently stirred for 1 h at 4° C. and then centrifuged at 100,000×g for 30 min at 4° C. The supernatant was diluted to 100 mM and cleared by centrifugation at 100,000×g for 30 min. The nuclear extract was applied to a DE-52 column equilibrated with buffer A (20 mM Hepes (pH 7.9), 1 mM EDTA, 10% glycerol, 1 mM DTT) containing 0.1 M KCl. Proteins were eluted from the column with buffer A containing 0.2 M KCl. Active fractions were pooled and subsequently applied to a DNA-Cellulose (Native DNA) column equilibrated with buffer A containing 0.2 M KCl. The column was washed with buffer A containing 0.5 M KCl and eluted with buffer A containing 0.7 M KCl. The active fractions were pooled and (NH₄)₂SO₄ was added to a final concentration of 0.436 g/ml and stirred for 30 min at 4° C. After centrifugation at 100,000×g for 20 min at 4° C., the precipitate was resuspended in buffer A containing 50 mM KCl and competitor DNA's (poly(dI-dC) and CATCAGGCCATCTGGCCCCTTGTTATTAA (SEQ ID NO: 1) at 50 μg/mL and 10 μg/mL, respectively). The sample was applied to a DNA-affinity column displaying double-stranded CATGGGCGCACGGGGCACTCCCGTGGTTCC DNA (SEQ ID NO:2). The column was washed with buffer A containing 0.4 M KCl and nonspecific competitor DNA's and eluted with buffer A containing 0.8 M KCl. The DNA affinity column step was repeated. The active fractions were pooled, resolved by SDS/PAGE and visualized by silver staining. The 100 kD band was excised from PVDF membrane, digested and individual peptides were sequenced as described in (Wang et al (1999) Cell 96, 771-784). Four peptides were found to match human AF156603.

B. Results

ChREBP was purified to homogeneity from the livers of rats fed a high carbohydrate diet and shown to consist of a single polypeptide with an apparent molecular weight of 100 kDa. The amino acid sequences of four peptides generated by tryptic digestion were determined. Each peptide was found to match the Williams-Beuren syndrome critical region 14 protein (WBSCR14), encoded by one of the 17 genes heterozygously deleted from patients suffering from the neurodevelopmental disorder Williams-Beuren syndrome. ChREBP is a member of the basic helix-loop-helix leucine zipper (bHLH-ZIP) family of transcription factors known to recognize E-box motifs within their target promoters.

### Example 2

### ChREBP Binding Activity is Induced in Rats Fed With a High Carbohydrate Diet

A. Materials and Methods

Gel shift assay. Gel mobility shift assays were performed as described by Liu et al. (Liu et al. (1993) J Biol Chem 268, 12787-12795). Double-stranded oligonucleotides (Table 1) were prepared by mixing equal amounts of the complementary single-stranded DNAs in 50 mM NaCl, heating to 70° C. for 15 min, and cooling to room temperature. The annealed oligonucleotides were labeled with ³²P in the presence of [γ³²P]-ATP (Amersham Pharmacia) and T4 polynucleotide kinase (New England Biolabs). Binding reactions were carried out in reaction mixture containing 20 mM Hepes (pH 7.9), 50 mM KCl, 5 mM DTT, 0.2 mM EDTA, 0.5 mM PMSF, 10% glycerol, 1 μg/μl of poly(dI-dC) (Amersham Pharmacia) and 1 μg/μl of nuclear extract. The reaction mixture was incubated at room temperature for 30 min and DNA-protein complexes were separated by electrophoresis in a 4.5% nondenaturing polyacrylamide gel.

Rats were starved for 48 h and subsequently re-fed a high fat diet for 24 h or a high carbohydrate diet for 24 h. Rat livers nuclear extracts were incubated with a radiolabeled oligonucleotide corresponding to the wild type LPK ChRE. Nuclear extracts were prepared as described in Example 1.

TABLE 1

The sequences of the wildtype (WT) and
mutated LPK ChRE's

| | |
|---|---|
| WT | |
| 5′-GGGCGCACGGGGCACTCCCGTGGTTCC-3′ | SEQ ID NO:3 |
| 3′-CCCGCGTGCCCCGTGAGGGCACCAAGG-5′ | SEQ ID NO:4 |
| M1 | |
| 5′-GGGCGaACGGGGCACTCCCGTtGTTCC-3′ | SEQ ID NO:5 |
| 3′-CCCGCtTGCCCCGTGAGGGCAaCAAGG-5′ | SEQ ID NO:6 |
| M2 | |
| 5′-GGGCGCcCGGGGCACTCCCGgGGTTCC-3′ | SEQ ID NO:7 |
| 3′-CCCGCGgGCCCCGTGAGGGCcCCAAGG-5′ | SEQ ID NO:8 |
| M3 | |
| 5′-GGGCGCAaGGGGCACTCCCtTGGTTCC-3′ | SEQ ID NO:9 |
| 3′-CCCGCGTtCCCCGTGAGGGaACCAAGG-5′ | SEQ ID NO:10 |
| M4 | |
| 5′-GGGCGCACtGGGCACTCCaGTGGTTCC-3′ | SEQ ID NO:11 |
| 3′-CCCGCGTGaCCCGTGAGGtCACCAAGG-5′ | SEQ ID NO:12 |
| M5 | |
| 5′-GGGCGCACGtGGCACTCaCGTGGTTCC-3′ | SEQ ID NO:13 |
| 3′-CCCGCGTGCaCCGTGAGtGCACCAAGG-5′ | SEQ ID NO:14 |
| M6 | |
| 5′-GGGCGCACGGtGCACTaCCGTGGTTCC-3′ | SEQ ID NO:15 |
| 3′-CCCGCGTGCCaCGTGAtGGCACCAAGG-5′ | SEQ ID NO:16 |
| M3/5 | |
| 5′-GGGCGCAtGcGGCACTCgCaTGGTTCC-3′ | SEQ ID NO:17 |
| 3′-CCCGCGTaCgCCGTGAGcGtACCAAGG-5′ | SEQ ID NO:18 |
| S − 1 | |
| 5′-GGGCGCACGGGGCCTCCCGTGGTTCCT-3′ | SEQ ID NO:19 |
| 3′-CCCGCGTGCCCCGGAGGGCACCAAGGA-5′ | SEQ ID NO:20 |
| S + 1 | |
| 5′-GGGCGCACGGGGCACTTCCCGTGGTTCC-3′ | SEQ ID NO:21 |
| 3′-CCCGCGTGCCCCGTGAAGGGCACCAAGG-5′ | SEQ ID NO:22 |

The E boxes are underlined in bold. Mutations within the E-box are indicated by lower case letters. S−1 and S+1 indicate the deletion or addition, respectively, of one base between the two E-boxes.

B. Results

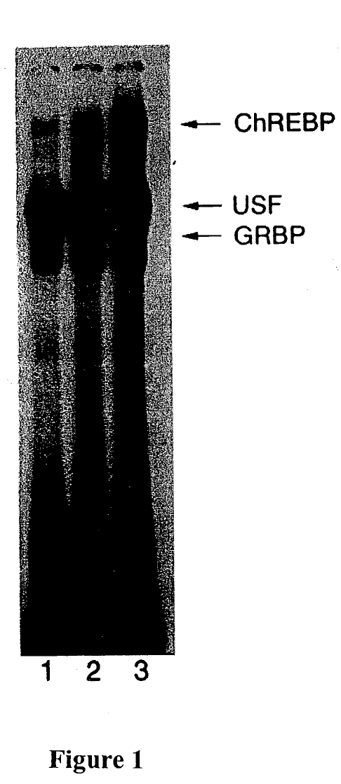

The carbohydrate response element (ChRE) identified within the promoter of the LPK gene contains two imperfect E-box motifs in a head to tail orientation separated by 5 base pairs (WT; Table 1). This sequence was used to examine DNA-binding activity in liver nuclear extracts prepared from rats starved for 48 h (FIG. 1, lane 1), fed either a high carbohydrate following starvation (lane 2) or high fat diet for 24 hours following starvation (lane 3). Arrows indicate the positions of the DNA-binding complexes containing ChREBP, upstream stimulatory factor (USF) or GRBP as determined by super-shift of the DNA-binding complex with antibodies specific for each protein. ChREBP binding to ChRE was enriched 2-3 fold only in rats fed the high carbohydrate diet in comparison to DNA-binding complexes containing USF or GRBP (FIG. 1A, lanes 2 and 3).

### Example 3

### ChREBP's DNA-binding Specificity In Vitro Correlates with Promoter Activity In Vivo.

A. Methods

Primary Hepatocytes Culture and Transfection. The construction of luciferase reporter plasmids was described in (Hasegawa et al. (1999) J. Biol Chem 274, 1100-1107). The mouse ChREBP coding region (Accession #AF156604) was amplified by PCR and ligated into the pcDNA3/V5HisB expression vector (Invitrogen).

Primary hepatocytes were prepared from male Sprague-Dawley rats using the collagenase (Life Technologies, Inc.) perfusion method (Berry and Friend (1969) J Cell Bio 143, 506-520) and plated in collagen-coated six-well tissue culture plates (Primaria Falcon, Franklin Lakes, N.J.) at a density of 1×10⁶ cells/well in glucose-free Dulbecco's modified Eagle's medium (Life Technologies, Inc.) supplemented with 100 nM dexamethasone, 10 nM insulin, 100 units/ml penicillin, 100 μg/ml streptomycin and 10% dialyzed fetal bovine serum (Life Technologies, Inc.). Following attachment, synthetic liposomes were used to transfect hepatocytes with 0.5 μg of the luciferase reporter plasmid and 0.1 μg of the internal control plasmid pRL-TK. After 14 h the media was replaced by Dulbecco's modified Eagle medium supplemented with 100 nM dexamethasone, 10 nM insulin, 10% Fetal Bovine Serum and either 10 mM lactate, 5.5 mM glucose or 27.5 mM glucose. The cells were incubated for an additional 12 h prior to determination of luciferase activity using the Dual-Luciferase Reporter Assay System (Promega).

Liver nuclear extracts and gel shift assays were performed as described in Examples 1 and 2.

B. Results

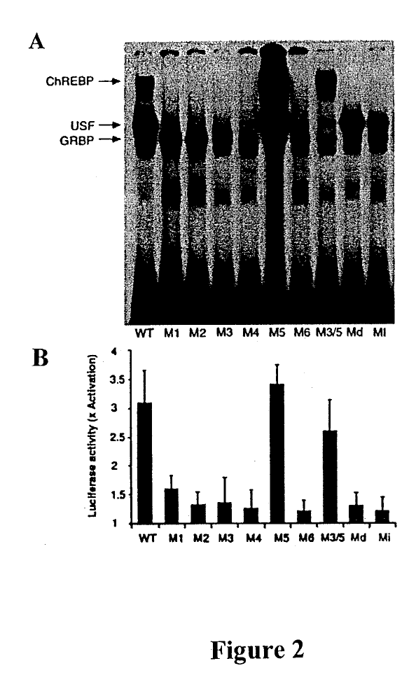

Mutant ChREs were designed with substitution mutations within the E-box motif, insertion or deletion of a single base between the 2 E-boxes (Table 1). Liver nuclear extracts from rats refed a high carbohydrate diet for 24 h following a 48 h starvation period were incubated with radiolabeled oligonucleotide corresponding to wild type and mutant ChRE oligonucleotides. The relative affinities of the observed DNA-binding proteins for the native LPK ChRE sequence and mutated variants (Table 1) are shown in the gel shift assay in FIG. 2A. Mutation of the modified E-box motifs to perfect palindromes (sequence M5) enhanced formation of the ChREBP DNA-binding complex while further deviation from the canonical E-box motif significantly impeded complex formation (sequences Ml-4, M6).

Complex formation between the variant sequences and wild type ChREBP correlated precisely with the ability of the ChRE variants to mediate glucose-induced transcription of a luciferase reporter in primary hepatocytes (FIG. 2B). Primary hepatocytes were transfected with the reporter constructs and the fold-activation of luciferase expression in response to high glucose (27.5 mM) was determined. Values represent the mean±standard error of four experiments. The agreement between ChREBP's DNA-binding specificity and the relative ability of the various DNA sequences to confer carbohydrate-responsive transcriptional activation indicate that ChREBP is a glucose-regulated transcription factor.

### Example 4

### ChREBP Transcriptional Activity is Limited to the Liver

A. Methods

Message levels were visualized from total RNA (25 μg) with a [³²P]-radiolabeled probe derived from the ChREBP cDNA. Nuclear extracts were prepared from rats refed a high carbohydrate diet for 24 h following 48 h starvation and incubated with the [³²P]-radiolabeled WT oligonucleotide (Table 1) followed by gel-shift analysis.

B. Results

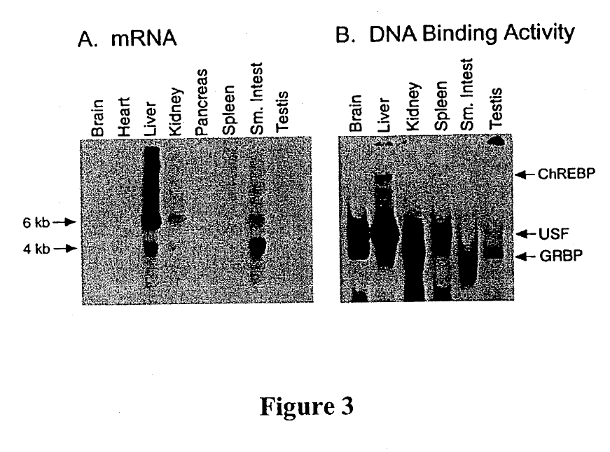

Northern analysis identified two ChREBP isoforms (6 kb and 4 kb) present in the liver, kidney, and small intestine (FIG. 3A). These results are consistent with the ChREBP expression pattern observed in the mouse (deLuis et al. (2000) Eur J Hum Genet 8, 215-222). However, gel-shift analysis of nuclear extracts prepared from various tissue sources demonstrated that ChREBP DNA-binding activity is only observable in liver nuclear extracts (FIG. 3B). Arrows indicate the positions of the DNA-binding complexes containing USF or GRBP as determined by super-shift of the DNA-binding complex with antibodies specific for each protein. The failure to detect the ChREBP mRNA in the pancreas is likely due to the fact that P cells represent only 1% of the cells in this organ. Conversely, the USF-containing complex was observed in many tissues that do not express LPK, as expected from USF's ubiquitous expression pattern (Sirito et al (1994) Nucleic Acids Res 22, 427-433). Although several other unidentified complexes are observed, only the ChREBP-containing complex is induced by a high-carbohydrate diet and is specific to the site of LPK expression.

### Example 5

### ChREBP Activates Transcription from the LPK Promoter in Response to Glucose in Hepatocytes.

A. Methods

Cell culture, transfection and luciferase assays were performed as described in Example 3.

Primary hepatocytes were cotransfected with 0.5 μg of the luciferase reporter construct driven by the LPK promoter and 1.5 μg of the ChREBP cDNA under the control of the constitutively active CMV promoter or with the empty expression vector. Following transfection, the cells were incubated for 12 h in media containing 10 mM lactate, 5.5 mM glucose or 27.5 mM glucose.

B. Results

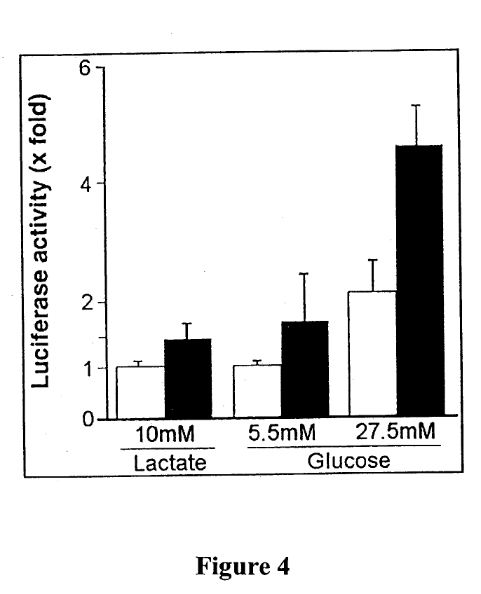

To determine if ChREBP can mediate glucose-activated transcription in cells, primary hepatocytes were co-transfected with the luciferase reporter gene driven by a 200 base-pair fragment of the LPK promoter containing the ChRE (Hasegawa et al. (1999) J. Biol Chem 274, 1100-1107) and an expression vector featuring ChREBP under the control of the constitutively active CMV promoter. When transfected cells were cultured in the absence of glucose (10 mM lactate) or in the presence of low glucose (5.5 mM), expression of the ChRE-driven reporter remained low and was unresponsive to forced overexpression of ChREBP (FIG. 4, dark bars). Supplementation of the culture medium with 27.5 mM glucose did lead to a two-fold induction of reporter expression in cells transfected with empty vector (FIG. 4, open bars). Values in FIG. 4 represent the mean±standard error of five experiments. This reporter induction reflects the presence of the endogenous glucose-responsive transcription factor that recognizes the LPK ChRE. Forced expression of ChREBP was able to further induce expression of the reporter an additional two-fold above the endogenous levels. The modest level of reporter induction, above endogenous ChREBP activity, in response to glucose is typical of the glucose-responsive transcriptional induction of many glycolytic enzymes in vivo. These results indicate that ChREBP may function as a glucose-responsive transcription factor in vivo.

### Example 6

### ChREBP's DNA-Binding Activity is Inhibited by Phosphorylation In Vitro.

A. Methods

Purified ChREBP was incubated with PKA and/or PP2A in vitro and measured by gel-shift analysis with the [³²P]-radiolabeled WT oligonucleotide. Purified ChREBP was incubated for 20 min with ATP (1 mM), PKA (0.1 U/μl), 0.2 U/μl PKI, 0.025 U/μl PP2A and the PP2A inhibitor, okadaic acid (10 nM).

B. Results

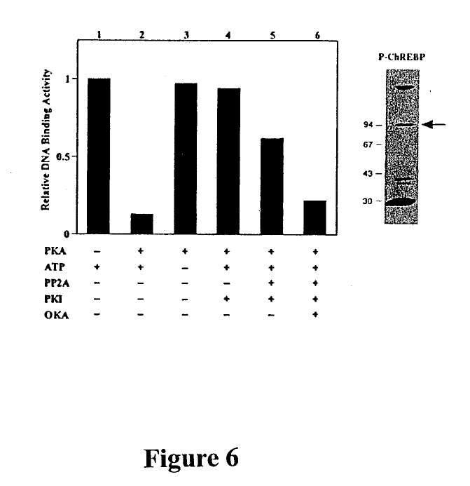

Purified ChREBP was incubated with the catalytic subunit of PKA in the presence of ATP resulting in phosphorylation of ChREBP and a 90% reduction in its DNA-binding activity (FIG. 6). Incubation of active ChREBP (lane 1) with both ATP (1 mM) and PKA (0.1 U/μl) abolished DNA-binding activity (lane 2) as measured by gel-shift analysis with the [³²P]-radiolabeled wild type oligonucleotide.

ChREBP inactivation required ATP and was inhibited by addition of a protein kinase inhibitor, PKI (FIG. 6, lane 4). DNA-binding activity of inactivated ChREBP was restored by the addition of the catalytic subunit of protein phosphatase 2A (PP2A) (FIG. 6, lane 5). Reactivation of phosphorylated ChREBP by PP2A is blocked by the phosphatase inhibitor, okadaic acid (FIG. 6, lane 6). Incubation of the protein with γ-[³²P]-ATP in the presence of PKA leads to radiolabeling of ChREBP observed following SDS-PAGE (FIG. 6, right panel). These data are consistent with the model that ChREBP activity is regulated by changes in its phosphorylation state.

### Example 7

### NLS and bHLH Regions are Required for ChREBP Glucose-responsive Transcriptional Activity.

A. Methods

Plasmids, Domain Deletion and Mutagenesis—The constructs were verified by nucleotide sequencing. Full-length mouse wild type (WT)-ChREBP cDNA (Accession # AF156604) was ligated into the Invitrogen mammalian expression vector pcDNA3 (ChREBP/pcDNA3) (Invitrogen, Carlsbad, Calif.) or Clontech vector pEGFP-N3 (ChREBP/pEGFP) (Clontech, Palo Alto, Calif.) encoding enhanced green fluorescent protein (GFP). The promoter region between positions −206 and −7 of the L-PK gene was ligated into the luciferase expression plasmid, pGL-3 basic vector (Promega) as previously described (Hasegawa et al. (1999) J. Biol Chem 274, 1100-1107). A pGL3 basic plasmid (SV40 promoter driving firefly luciferase gene), carrying the promoter region between positions −206 and −7 of the L-PK gene and pRL-TK (thymidine kinase promoter driving renilla luciferase gene) were also transfected into each cell as a reporter gene and an internal control, respectively. Plasmids containing the NLS, PRO, bHLH/ZIP, ZIP-like deletion mutants, or point mutants in the putative phosphorylation sites of PKA of ChREBP were constructed using QuickChange Site-Directed Mutagenesis Kit (STRATAGENE). Oligonucleotides used to introduce a new restriction site immediately upstream or downstream of each domain are listed in Table 2. Oligonucleotides used to introduce the desired mutations are listed in Table 3. The double mutant plasmids were constructed using the same method as that used for making single mutants.

Rat primary cultured hepatocytes were transfected with a plasmid comprising a gene encoding the wild type-ChREBP or ChREBP with various domain deletion mutants. After transfection, cells were incubated under 5.5 mM (□) or 27.5 mM (▪) glucose for 12 h. Relative luciferase activity was calculated and expressed as mean±SE (n=5). *P<0.05 compare to that of wild type-ChREBP.

TABLE 2

Oligonucleotides used to construct plasmids containing deletions of domain

| | | | | |
|---|---|---|---|---|
| Deletion | | Restr. | | |
| Domain | Location | Site | Orientation | Sequence (Position) |

| | | | | | | |
|---|---|---|---|---|---|---|
| NLS | Upstream | DraI | Forward | 5′-CACATGAGCACTTTAAACCTGAGGCTGT-3′ | (bp461-488) | (SEQ ID NO:23) |
| | | | Reverse | 5′-ACAGCCTCAGGTTTAAAGTGCTCATGTG-3′ | (bp461-488) | (SEQ ID NO:24) |
| | Downstream | MscI | Forward | 5′-TGGTGATGCGCTGGCCACACGTGTGGAG-3′ | (bp527-554) | (SEQ ID NO:25) |
| | | | Reverse | 5′-CTCCACACGTGTGGCCAGCGCATCACCA-3′ | (bp527-554) | (SEQ ID NO:26) |
| Proline- | Upstream | AflII | Forward | 5′-TTCCTGAAGACCTTAAGACCAAGATCCC-3′ | (bp1148-1175) | (SEQ ID NO:27) |
| rich | | | Reverse | 5′-GGGATCTTGGTCTTAAGGTCTTCAGGAA-3′ | (bp1148-1175) | (SEQ ID NO:28) |
| | Downstream | AflII | Forward | 5′-TCTCTGTAGACCTTAAGCCCCATGGGTA-3′ | (bp1418-1445) | (SEQ ID NO:29) |
| | | | Reverse | 5′-TACCCATGGGGCTTAAGGTCTACAGAGA-3′ | (bp1418-1445) | (SEQ ID NO:30) |
| bHLH/ | Upstream | AflII | Forward | 5′-ACAACAAGATGCTTAAGCGACGTATCAC-3′ | (bp1967-1994) | (SEQ ID NO:31) |
| ZIP | | | Reverse | 5′-GTGATACGTCGCTTAAGCATCTTGTTGT-3′ | (bp1967-1994) | (SEQ ID NO:32) |
| | Downstream | AflII | Forward | 5′-TCAACTTGTGCCTTAAGCAGCTACCGGC-3′ | (bp2219-2246) | (SEQ ID NO.33) |
| | | | Reverse | 5′-GCCGGTAGCTGCTTAAGGCACAAGTTGA-3′ | (bp2219-2246) | (SEQ ID NO:34) |
| ZIP-like | Upstream | DraI | Forward | 5′-CCGCCAGCTTGTTTAAACTCCGCCAGAC-3′ | (bp2399-2426) | (SEQ ID NO:35) |
| | | | Reverse | 5′-GTCTGGCGGAGTTTAAACAAGCTGGCGG-3′ | (bp2399-2426) | (SEQ ID NO 36) |
| | Downstream | MscI | Forward | 5′-CCAGCCTTGTGTGGCCACAAGCCACACG-3′ | (bp2531-2558) | (SEQ ID NO 37) |
| | | | Reverse | 5′-CGTGTGGCTTGTGGCCACACAAGGCTGG-3′ | (bp2531-2558) | (SEQ ID NO:38) |

Underlined letters indicate cleavage sites for restriction endonucleases used.

TABLE 3

Oligonucleotides used to construct plasmids containing point
mutations in the putative phosphorylation sites of PKA

| | | |
|---|---|---|
| Mutant | Orientation | Sequence (Position) |

| | | | | |
|---|---|---|---|---|
| S196A | Forward | 5′-CTCCGTAAGTCCGCCAGGGAAGGGGAT-3′ | (bp574-600) | (SEQ ID NO:39) |
| | Reverse | 5′-ATCCCCTTCCCTGGCGGACTTACGGAG-3′ | (bp574-600) | (SEQ ID NO:40) |
| S196D | Forward | 5′-CTCCGTAAGTCCGACAGGGAAGGGGAT-3′ | (bp574-600) | (SEQ ID NO:41) |
| | Reverse | 5′-ATCCCCTTCCCTGTCGGACTTACGGAG-3′ | (bp574-600) | (SEQ ID NO:42) |
| S626A | Forward | 5′-GAGCGGCGACTAGCCGGGGATCTCAAC-3′ | (bp1864-1890) | (SEQ ID NO:43) |
| | Reverse | 5′-GTTGAGATCCCCGGCTAGTCGCCGCTC-3′ | (bp1864-1890) | (SEQ ID NO:44) |
| S626D | Forward | 5′-GAGCGGCGACTAGACGGGGATCTCAAC-3′ | (bp1864-1890) | (SEQ ID NO 45) |
| | Reverse | 5′-GTTGAGATCCCCGTCTAGTCGCCGCTC-3′ | (bp1864-1890) | (SEQ ID NO:46) |
| T66A | Forward | 5′-AACCGACGTATCGCCCACATCTCCGCG-3′ | (bp1981-2007) | (SEQ ID NO.47) |
| | Reverse | 5′-CGCGGAGATGTGGGCGATACGTCGGTT-3′ | (bp1981-2007) | (SEQ ID NO.48) |
| T666D | Forward | 5′-AACCGACGTATCGACCACATCTCCGCG-3′ | (bp1981-2007) | (SEQ ID NO:49) |
| | Reverse | 5′-CGCGGAGATGTGGTCGATACGTCGGTT-3′ | (bp1981-2007) | (SEQ ID NO 50) |

The mutated nucleotides are underlined.

B. Results

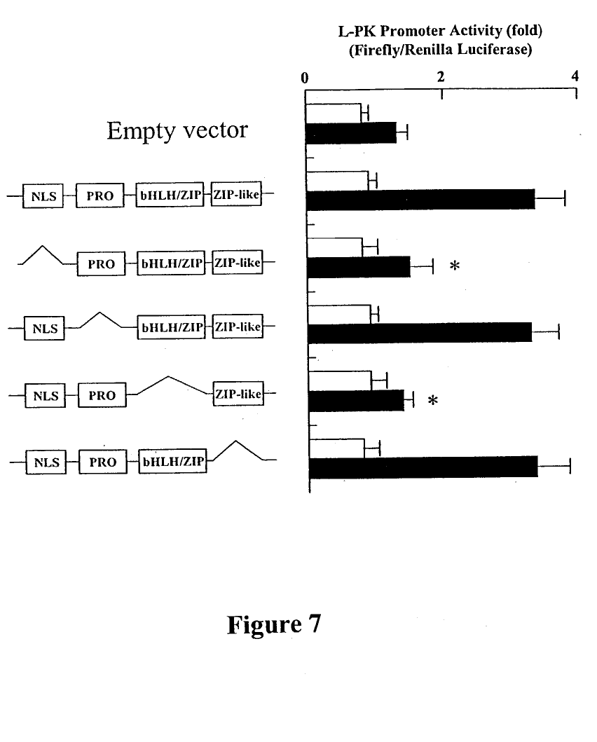

Various domain deletion mutants of ChREBP were prepared. The effects of these mutant ChREBPs on L-PK transcription activity were determined with a dual-luciferase reporter system. Rat primary cultured hepatocytes were transfected with the WT- and mutant ChREBPs, and the cells were maintained in low (5.5 mM, □ and high glucose (27.5 mM, ▪ for 12 h. The transcriptional activities in the cells transfected with empty vector were due to the endogenous activity of ChREBP in the primary hepatocytes. The WT-ChREBP showed at least 2-fold activation of the activity in high glucose compared to that in the empty vector (FIG. 7). To confirm this increase was due to glucose metabolism, and not osmotic stress, primary hepatocytes were incubated with 500 mM NaCl instead of 27.5 mM glucose. The increase in transcriptional activity was not seen in NaCl, and the transcriptional activation required high glucose.

Among the mutant ChREBPs, deletion of NLS or the bHLH/Zip domain resulted in complete loss of the high glucose-induced transcriptional activation. However, the deletion of PRO or the Zip-like domain at the C-terminus did not affect the transcriptional activity. These results demonstrated that the NLS and bHLH/Zip domains were essential for the glucose response, but the PRO and the Zip-like domains were not involved in the high glucose-induced transcriptional activation of L-PK gene.

### Example 8

### Transcriptional Activity of ChREBP is Inhibited by PKA and Activated by PP2A.

A. Methods

Cell culture, transfection and luciferase assays were performed as described in Example 3.

Protein Kinase and Protein Phosphatase Inhibitors—Prior to exchange to 5.5 or 27.5 mM glucose medium, transfected primary culture hepatocytes were treated for 1 h with 10 μM H-89, 500 nM cantharidic acid (Calbiochem), 5 μM KN-62 (Calbiochem), 30 μM PD98059 (Calbiochem), 50 μM Genistein, or dimethylsulfoxide vehicle.

Rat primary cultured hepatocytes were transfected a plasmid comprising a gene encoding the wild type (WT)-ChREBP. H-89, cantharidic acid, or dimethylsulfoxide vehicle was added, and the cells were incubated in 5.5 mM (□) or 27.5 mM (▪) glucose for 12 h. Relative luciferase activity was calculated and expressed as mean±SE (n=5). *P<0.05 compared to that of WT-ChREBP.

B. Results

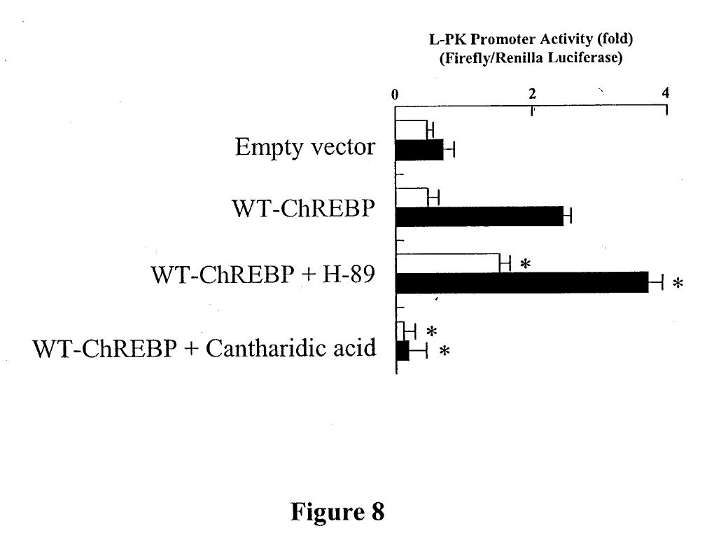

Protein phosphorylation and dephosphorylation is one way to regulate the activity of a transcription factor. To investigate the involvement of phosphorylation and dephosphorylation of ChREBP in the glucose-induced activation of the L-PK gene transcription, a number of different inhibitors of various protein kinases and protein phosphatases were added to the culture medium. The addition of H-89, a specific inhibitor of PKA, to the culture medium increased the transcriptional activity in the ChREBP-overexpressed hepatocytes under both low and high glucose (FIG. 8). H-89 also stimulated endogenous ChREBP but the effect was small. On the other hand, cantharidic acid, a specific inhibitor of PP2A, inhibited transcriptional activity of the L-PK gene in both 5.5 and 27.5 mM glucose. KN-62, PD98059, and Genistein, which are specific inhibitors of calcium/calmodulin-dependent protein kinase II, mitogen-activated protein kinase, and tyrosine kinase, respectively, did not affect transcriptional activity of the L-PK gene, suggesting that these protein kinases were not involved in the regulation of ChREBP. These results suggested that ChREBP activity was inhibited by phosphorylation catalyzed by PKA, and the activity was regained by PP2A.

### Example 9

### Subcellular Localization of ChREBP-GFP Fusion Protein

A. Methods

Cell culture, transfection and luciferase assays were performed as described in Example 6.3. Plasmids were generated as described in Example 7.

Determination of Subcellular Localization of ChREBP—Subcellular localization of GFP-fused WT-ChREBP or its mutants was determined using a confocal laser scanning microscope (Bio Rad, Hercules, Calif.). In order to quantitate nuclear localization of ChREBP, for each condition, 100 transfected hepatocytes from 5 independent experiments were scored in a blinded fashion as to whether the GFP-fused ChREBP was predominantly nuclear or cytoplasmic. The identity of the nucleus was verified by comparative phase-contrast microscopy. B. Results

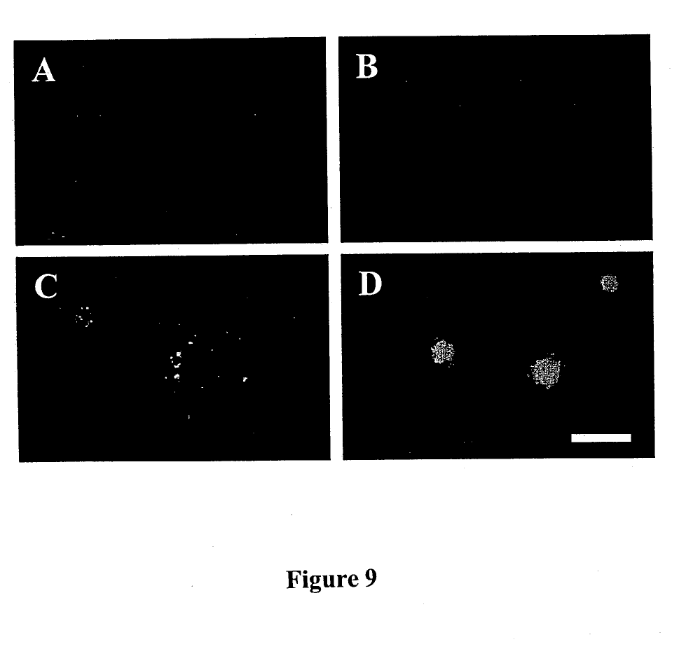

NLS domains serve as signals for import of transcription factors into nuclei. Plasmid comprising a gene encoding the GFP-fused WT-ChREBP and a series of domain-deletion mutants of ChREBP were generated, transfected into primary cultured hepatocytes, and observed for ChREBP subcellular localization with confocal laser scanning microscopy. FIG. 9 shows representative images of subcellular localization of ChREBP under low or high glucose. Bar in FIG. 9 =10 μm. Nontransfected hepatocytes did not show any fluorescence (FIG. 9A), and endogenous ChREBP could not be seen under these conditions. Empty vector-transfected hepatocytes showed weak diffused fluorescence throughout the cells (FIG. 9B). The GFP-fused wild type ChREBP-transfected hepatocytes showed cytoplasmic (FIG. 9C) and nuclear localization (FIG. 9D) in low and high glucose, respectively. The nuclear import was complete in 8 hr, while the L-PK promoter activity took 20 hr. Thus, the rate of the translocation of ChREBP was rapid enough to activate the L-PK gene expression.

Rat primary cultured hepatocytes were transfected with a plasmid comprising a gene encoding the GFP-fused wild type-ChREBP or NLS domain deletion mutants. H-89, cantharidic acid, or dimethylsulfoxide vehicle was added, and the cells were incubated under 5.5 mM (□) or 27.5 mM (▪) glucose for 12 h. The values are expressed as mean±SE. *P<0.05 compared to that of wild type-ChREBP.

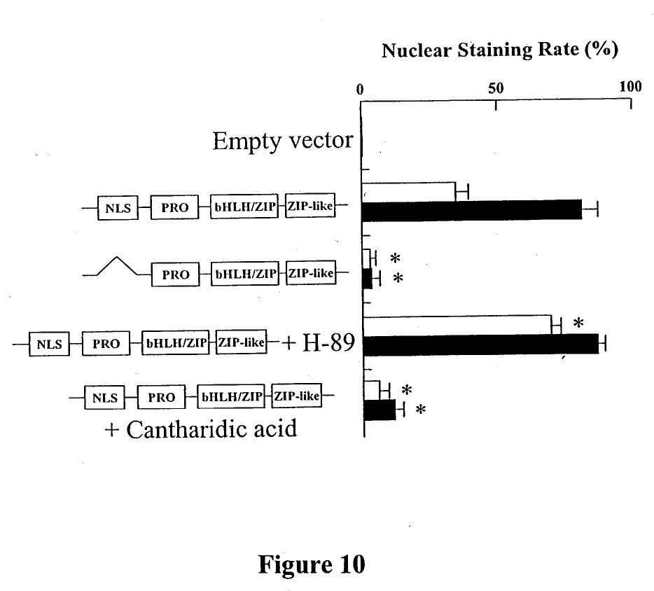

The WT-ChREBP showed about 40% and 80% of nuclear staining rate in 5.5 mM and 27.5 mM glucose, respectively (FIG. 10). NLS-deleted ChREBP mutant showed less than 10% of nuclear staining rate in both 5.5 mM (□) and 27.5 mM (▪) glucose. These results demonstrated that high glucose stimulated the translocation of ChREBP from cytosol into nuclei. By treatment of hepatocytes with H-89, nuclear staining rate was increased in 5.5 mM glucose. On the other hand, cantharidic acid significantly decreased the nuclear staining rate, suggesting that dephosphorylated ChREBP, but not the phosphorylated ChREBP, was translocated into the nuclei (FIG. 10A). Thus, NLS domain was required for nuclear translocation and the nuclear translocation of ChREBP was regulated negatively by PKA catalyzed phosphorylation and positively by dephosphorylation by PP2A.

### Example 10

### Subcellular Localization of ChREBP Mutants

A. Methods

Cell culture, transfection and luciferase assays were performed as described in Example 3. Plasmids were generated as described in Example 7. Microscopy was performed as described in Example 9.

B. Results

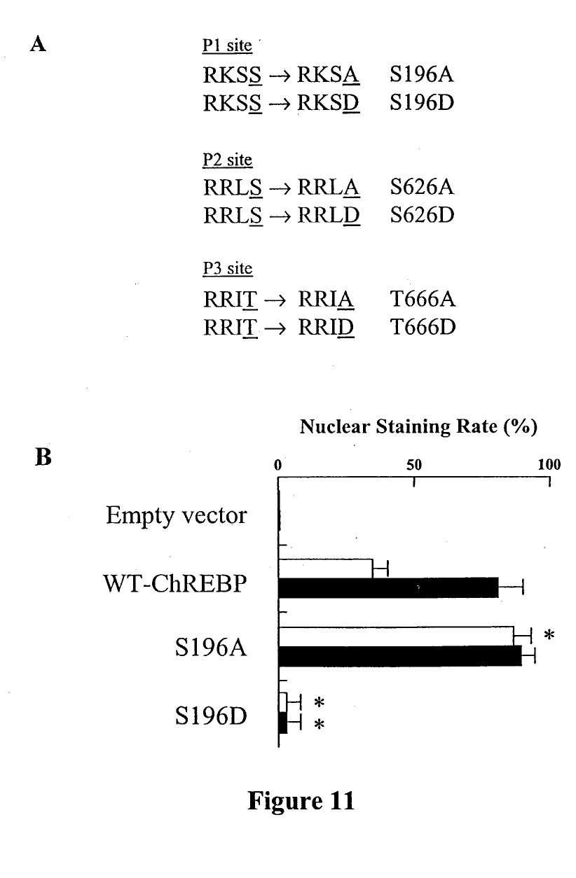

In order to determine the phosphorylation sites of ChREBP which regulate the nuclear import, all three consensus PKA phosphorylation sites were mutated. Since a putative PKA phosphorylation site (Ser¹⁹⁶ designated as P1) occurs near the NLS domain, substitutions were introduced (S196A and S196D; FIG. 11A) and the effect on nuclear localization was determined.

Rat primary cultured hepatocytes were transfected with plasmids comprising a gene encoding GFP-fused wild type (WT)-ChREBP, S196A, or S196D and the cells were incubated in 5.5 mM (□) or 27.5 mM (▪) glucose for 12 h (FIG. 11B). The values are expressed as mean±SE. *P<0.05 compared to that of WT-ChREBP.

S196A showed about 90% of nuclear staining rate in both 5.5 and 27.5 mM glucose (FIG. 11B), while S196D mutant showed less than 10% of nuclear staining rate (FIG. 11B). To investigate the effects of phosphorylation states of the other PKA phosphorylation sites, double mutants were constructed (S196A+S626A, S196A+S626D, S196A+T666A, and S196A+T666D)(FIG. 11A). All these double-mutants showed about 90% of nuclear staining rate, suggesting strongly that the nuclear translocation was regulated by phosphorylation at Ser¹⁹⁶, regardless of the phosphorylation states of the other sites.

### Example 11

### Dibutyryl (db)-cAMP Inhibits ChREBP Binding in vivo

A. Methods

Treatment of Rats with Dibutyryl (db)-cAMP and Nuclear Extract Preparation—Male Sprague-Dawley rats weighting 250 g were used in all experiments. After 48 h starvation, the rats were fed a high carbohydrate diet and db-cAMP (1 mmol/kg) was injected intraperitoneally. The rats were sacrificed 10 min and 30 min after injection and nuclear extracts were prepared immediately from rat liver as described in Example 1. Gel shifts were performed as described in Example 2.

B. Results

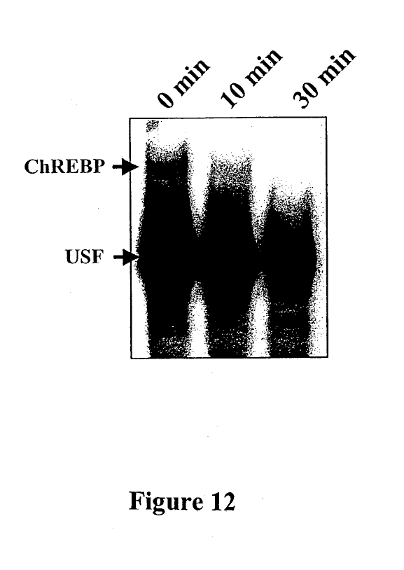

To determine the effect of cAMP on the DNA-binding activity of ChREBP in vivo, rats were treated with intraperitoneal injection of db-cAMP. Nuclear extracts of db-cAMP-treated rat livers were assayed for DNA-binding activity. The DNA-binding activity was readily inactivated within 10 min after the db-cAMP injection (FIG. 12). Since these rats were fed, ChREBP was already localized in nuclei. In preliminary experiments using GFP-ChREBP, ChREBP was not exported from nuclei to cytoplasm within 10 min after addition of db-cAMP into 27.5 mM glucose medium (n=3; data not shown). It has also been reported that nuclear export takes 30 min to 2 h (Efthymiadis et al (1998) Biochem Biophys 355, 254-261; Mao et al (1998) J Biol Chem 273, 23621-23624.). The loss of the DNA-binding activity maybe due to inactivation of DNA-binding, but the possibility of export of ChREBP cannot be excluded.

### Example 12

### Effects of PKA and PP2A on the DNA-binding Activity of ChREBP In Vitro.

A. Methods

Recombinant Protein Production—Recombinant protein of mouse ChREBP was expressed in Escherichia coli by inserting the C-terminus of the ChREBP construct, encoding the amino acids 651-864 of ChREBP+His₆ tag, into the BamHI and HindIII sites of the T7-based expression vector pMal-c2 (New England Bio Lab, Beverly, Mass.) to produce an N-terminal fusion with maltose-binding protein. Expressed protein was purified according to manufacturer's instruction.

Phosphorylation and Dephosphorylation of ChREBP—The reaction mixture contained, in a final volume of 0.2 ml, 50 mM Tris/Cl pH 7.5, 0.2 μM [γ-³²P]ATP (3000 Ci/mmol), 5 mM MgCl₂, 0.2 mM EDTA, 2 mM dithiothreitol, and 5 mM potassium phosphate. The reaction was initiated with the addition of catalytic subunit of PKA (200 units) or protein phosphatase 2A (PP2A, 200 units), and the reaction mixture was incubated at 30° C. At given time intervals, aliquots were removed and DNA-binding activity was determined by gel shift assay as described in Example 2. [³²P]phosphate-bound ChREBP was also determined as previously described (Kitajima and Uyeda (1983) J. Biol Chem 258, 7352-7357).

B. Results

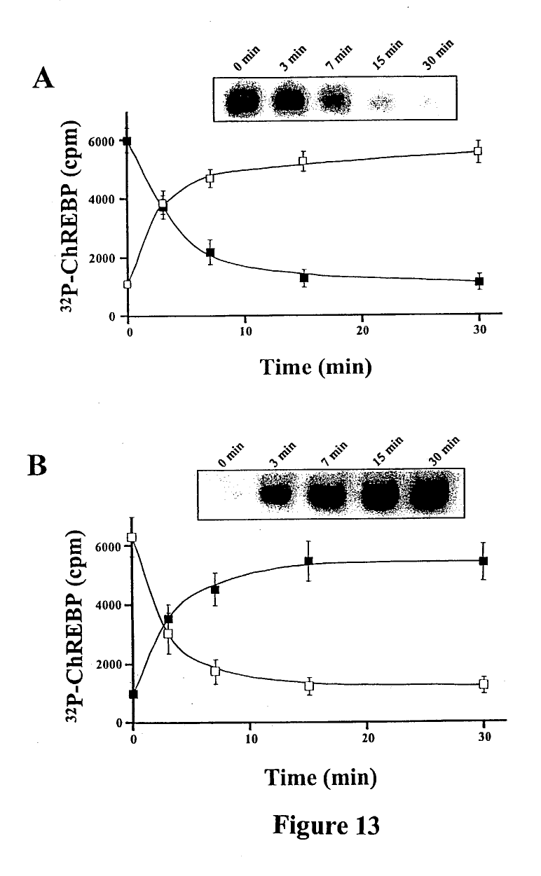

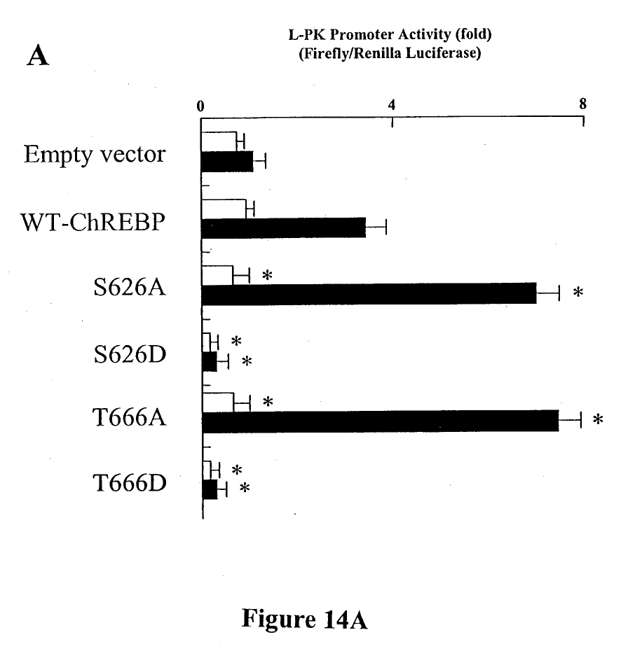

FIG. 13A is a graph showing that phosphate incorporation mediated by PKA correlates directly with DNA binding. Recombinant C-terminus ChREBP was incubated with PKA in the presence of [γ-³²P]ATP. At given time intervals, [³²P]phosphate incorporation (□) and DNA-binding activity (▪) were determined. Values are expressed as mean±SE. The insert shows a representative image for effects of PKA on the DNA-binding activity of ChREBP using gel shift assay. FIG. 13B shows the rates of dephosphorylation of ChREBP by PP2A and the activation of DNA-binding activity of ChREBP in vitro. Phosphorylated C-terminus ChREBP was incubated with PP2A. At given time intervals, remaining phosphorylated C-terminus ChREBP (□) and DNA-binding activity (▪) were determined. Values are expressed as mean±SE. The inserts shows a representative image for effects of PP2A on the DNA-binding activity of ChREBP.

Using a recombinant C-terminal region of ChREBP containing the bHLH/ZIP with a single phosphorylation site (Thr⁶⁶⁶, P3), the effect of PKA-dependent phosphorylation of Thr⁶⁶⁶ on the DNA-binding activity of bHLH/ZIP domain was determined. As shown in FIG. 13A and inset, ³²P was incorporated into the bHLH/ZIP domain and the DNA-binding activity was rapidly inactivated, and the rates of these changes were similar. The observation that both the phosphate incorporation and loss of the DNA-binding activity were similar suggested that the inactivation of DNA-binding was a direct result of the phosphorylation at Thr⁶⁶⁶. Furthermore, PKA-dependent phosphorylation of the bHLH/ZIP domain was fully reversible, since the incubation of the ³²P-phosphorylated bHLH/ZIP domain with catalytic subunit of PP2A resulted in complete recovering of the activity (FIG. 13B and inset).

### Example 13

### Effect of ChREBP Mutations at Ser⁶²⁶ and Thr⁶⁶ on Transcriptional Activity of L-PK Gene.

A. Methods

Cell culture, transfection and luciferase assays were performed as described in Example 3. Plasmids generated as described in Example 7.

B. Results

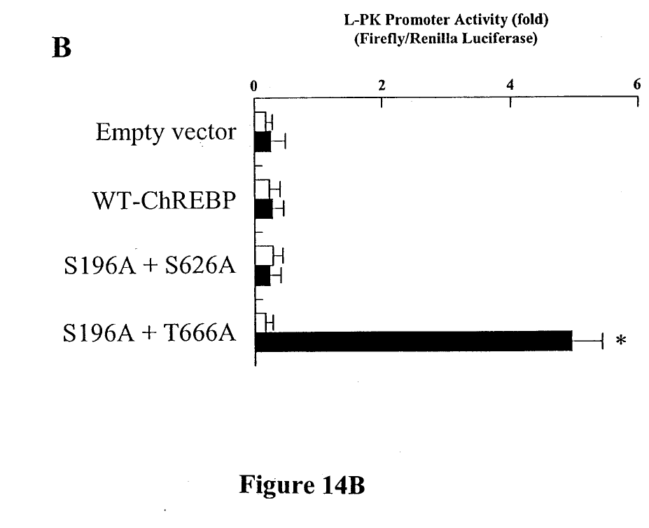

FIG. 14A shows that S626A and T666A mutants expression exhibit 2.5 fold activation of transcriptional activity in comparison to WT-ChREBP. Rat primary cultured hepatocytes were transfected with wild type (WT)-ChREBP, S626A, S626D, T666A, or T666D. After transfection, the cells were incubated in 5.5 mM (□) or 27.5 mM (▪) glucose for 12 h. Relative luciferase activity was calculated and expressed as mean±SE (n=5). *P<0.05 compared to that of WT-ChREBP. FIG. 14B shows that the effects of db-cAMP on the L-PK gene transcription in S626A- and T666A-double mutant expressed in hepatocytes demonstrate that Thr666 to be one of the key residues for DNA binding. Rat primary cultured hepatocytes were transfected with wild type (WT)-ChREBP, S196A+S626A, or S196A+T666A. After transfection, cells were incubated under 5.5 mM (□) or 27.5 mM (▪) glucose, and cAMP was added both low and high glucose. Relative luciferase activity was calculated and expressed as mean±SE (n=5). *P<0.05 compared to that of WT-ChREBP.

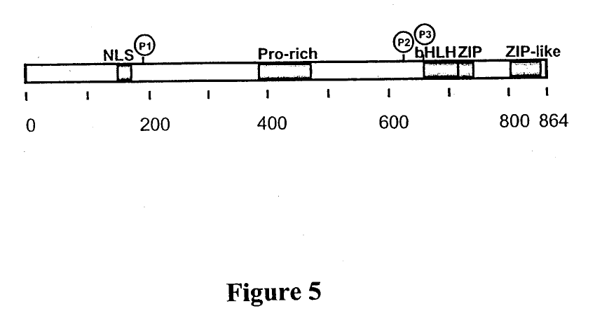

There are two PKA phosphorylation sites, Ser⁶²⁶ located near the bHLH domain and Thr⁶⁶⁶ situated within the basic residues of the bHLH domain (designated as P2 and P3, representing in FIG. 5). To determine which phosphorylation site regulates the ChREBP DNA-binding activity, substitution mutants were generated (S626A, S626D, T666A, and T666D mutants). Both S626A and T666A mutants showed about 2.5-fold activation of transcriptional activity by high glucose compared to that of WT-ChREBP (FIG. 14A). On the other hand, both S626D and T666D mutants showed much lower activity under both high and low glucose (FIG. 14A). These results indicated that both Ser⁶²⁶ and Thr⁶⁶⁶ were the sites of phosphorylation and involved in the inhibition of the DNA-binding activity. In order to determine which site was more crucial for the activity, we constructed two double mutants (S196A+S626A and S196A+T666A) and examined the effects of db-cAMP on transcriptional activity. The transcriptional activity of S196A+T666A, but not S196A+S626A, was not inhibited by treatment with db-cAMP (FIG. 14B), suggesting that Thr⁶⁶⁶ was the most important regulatory site, and Ser⁶²⁶ may only play a supportive role in the DNA-binding.

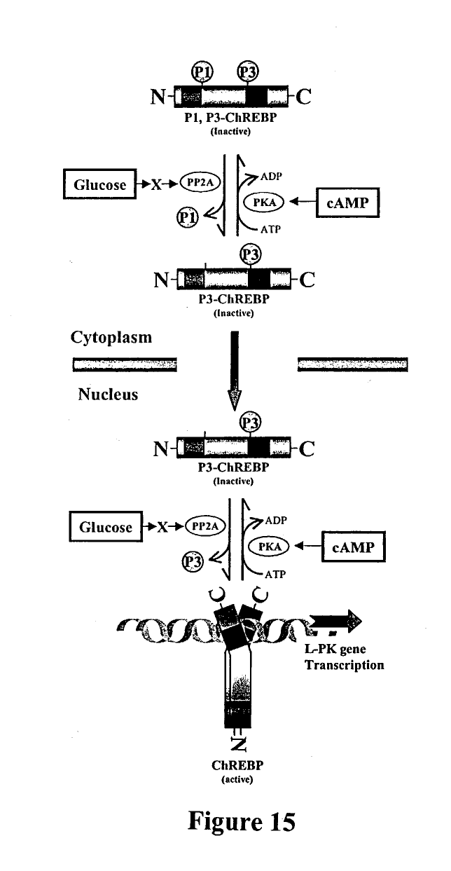

FIG. 15 is a schematic representation of regulatory mechanisms of ChREBP by glucose and cAMP in hepatocytes. Two PKA phosphorylation sites of ChREBP, Ser¹⁹⁶ (P1) and Thr⁶⁶⁶ (P3), play crucial roles in high glucose-induced activation of ChREBP (P1 and P3 indicate the phosphorylated form at the sites). Glucose signaling may activate PP2A via a metabolite (“X”). In cytoplasm, X-activated activated PP2A dephosphorylates PI site of the ChREBP, which results in stimulation of import of ChREBP into the nucleus. Once ChREBP is localized in the nucleus, glucose activates the inactive form of ChREBP (P3-ChREBP), by dephosphorylation of P3 site catalyzed by PP2A. ChREBP, which is dephosphorylated at P1 and P3 sites, binds to ChRE of L-PK gene, and consequently activates transcription of the L-PK gene.

### Example 14

### ChREBP Binding to Other Genes in Glycolysis and Lipogenesis

A. Methods

Gel shift assays were performed as described in Example 2. Sequences used as in Table 4.

TABLE 4

| | |
|---|---|
| Gene | Sequence |

| | | |
|---|---|---|
| L-PK | 5′-GGGCGCACGGGGCACTCCCGTGGTTC-3′ | (SEQ ID NO:51) |
| S14 | 5′-AGTTCTCACGTGGTGGCCCTGTGCTTGG-3′ | (SEQ ID NO:52) |
| InsZ | 5′-GACCTGTCCCTGCTCACAGCTGGGGGCAAATGTCTCCAGGAGAGCAAAGCC-3′ | (SEQ ID NO:53) |
| FAS | 5′-CTTCCTGCATGTGCCACAGGCGTGTCACCCTC-3′ | (SEQ ID NO:54) |
| ACL | 5′-GATCTGATGGGGGGCGGGGAGGAGCCCGGGATC-3′ | (SEQ ID NO:55) |
| ACC | 5′-CAAATGCCATGTGAAAACCCATAGCCTG-3′ | (SEQ ID NO:56) |

B. Results

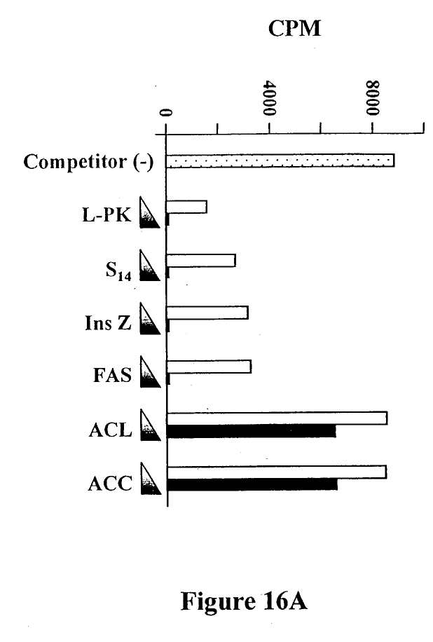

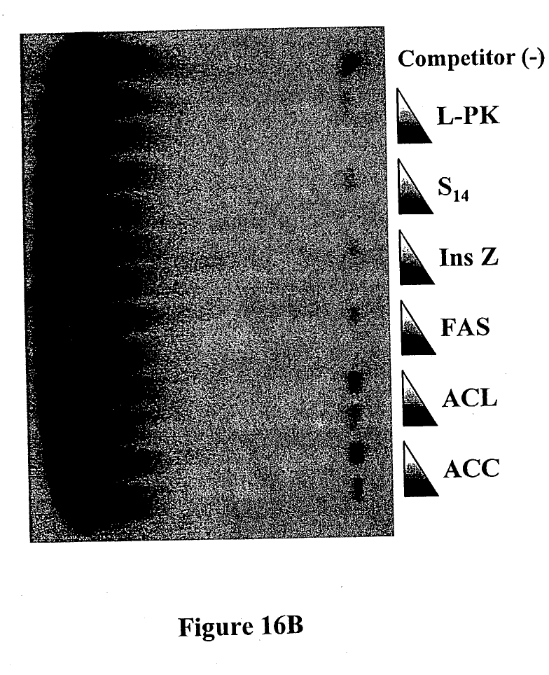

ChREBP binds to promoter regions of genes involved in lipogenesis and glucose metabolism, including L-type pyruvate kinase (L-PK), spot 14 (S14) protein, insulin (InsZ), fatty acid synthase (FAS), ATP citrate lyase (ACL) and acetyl CoA carboxylase (ACC) (FIGS. 16A and B).

### Example 15

### Feeding Studies Using ChREBP Knockout Animals

A. Methods—Generation of ChREBP −/− Mice.

Mice were produced using standard transgenic/knockout protocols. Briefly, the mouse ChREBP locus spanning all 17 exons has been sequenced (Accession # AC084109) and used to generate DNA fragments flanking exons 12 through 14. Substitution of this region with a selection marker for resistance to the antibiotic G418 resulted in the deletion of the bHLH domain. The targeting construct contains the herpes simplex virus thymidine kinase gene to provide negative selection against clones in which the cassette does not integrate via homologous recombination. Embryonic stem cells were transfected with the targeting construct. Following confirmation by Southern blot analysis, ES clones bearing the targeted disruption of the ChREBP gene were injected into mouse blastocytes to generate chimeric mice. Chimeric lines featuring germline transmission of the disrupted gene were mated to wild type mice to generate heterozygous mice at the ChREBP locus. These mice were subsequently crossed to generate mice homozygous for the targeted allele. These ChREBP knockout (−/−) mice were identified by genotyping DNA and the analyses were consistent with Mendelian inheritance. The ChREBP −/− mice appear normal at birth and during the past several weeks.

B. Results

ChREBP −/− mice were fed a high carbohydrate diet for three days and the mRNA levels of key enzymes were determined using RTPCR. Results of mRNA analysis is compared to levels of key enzymes in SREBP 1c −/− (steroid response element binding protein knockout) mice, which were also fed a high carbohydrate diet. The values shown in Table 5 below represent the relative mRNA levels of the same key enzymes in wild type mice which was arbitrarily defined as 1. Cyclophilin was used as the invariant control.

In ChREBP −/− mice, the mRNA of ACC, FAS, and malic enzymes were significantly repressed (Table 5). Levels of HMG CoA reductase were increased in comparison to wild type mice. Glucokinase and PEPCK mRNA also increased. These results suggest that lipogenesis was severely repressed in the ChREBP −/− mice. The reduction of malic enzymes suggests that NADPH necessary for fat synthesis was also repressed.

The results indicate that ChREBP −/− repressed expression of all the lipogenic enzymes genes including ATP citrate lyase, AcCoA carboxylase, fatty acid synthase, and stearoylCoA desaturase. In addition, reduction of malic enzyme suggests that NADPH necessary for fat synthesis was also repressed. Compared to the SREBP /− mice, the repression of the lipogenic genes was more severe in ChREBP, suggesting that ChREBP may play more important roles than SREBP in lipogenesis under high carbohydrate diet. The glucokinase and the PEPCK gene are increased but Glut2 and glut4 genes were repressed, suggesting that glucose metabolism is also affected.

Based on these results we can tentatively predict the phenotype which results from a ChREBP knockout. The knockout mice will have decreased blood sugar levels and decreased blood insulin levels. Indeed, the hepatic glucose concentration in the mice have decreased capacity for fat synthesis but increased ability to make steroids.

TABLE 5
mRNA levels of various enzymes involved in glucose
metabolism and lipogenesis from ChREBP−/− and SREBP-1c−/−
Mice Fed With High Carbohydrate Diet as measured by RT-PCR

| GENE | ChREBP−/− | SREBP1c−/− |
|---|---|---|
| Glucokinase | 1.51 | 0.41 |
| PEPCK | 2.91 | 2.88 |
| G-6-Pas | 0.01 | |
| Glut-2 | 0.31 | |
| Glut 4 | 0.12 | |
| SREBP 1c | 0.53 | 0.00 |
| AcCoA synthetase | 0.82 | 0.37 |
| ATP citrate lyase | 0.11 | 0.32 |
| AcCoA carboxylase (ACC) | 0.25 | 0.60 |
| Fatty acid synthase (FAS) | 0.09 | 0.25 |
| StearoylCoA desaturase 1 | 0.03 | 0.30 |
| Glycerol-3-P acyltransferase | 0.35 | 0.26 |
| Malic enzymes | 0.12 | 0.23 |
| Glc-6-P dehydrogenase | 0.68 | 0.25 |
| HMG CoA Synthase | 2.09 | 1.41 |
| HMG CoA reductase | 5.44 | 1.34 |
| LDLR | 1.38 | 0.92 |
| ApoB | 1.33 | 0.93 |
| ApoE | 1.52 | 0.95 |
| Cholesterol 7-alpha hydroxylase | 7.32 | |
| IRS 1 | 0.66 | 0.82 |
| IRS 2 | 0.61 | 1.73 |
| Cyclophilin | 1.00 | |

### Example 16

### Role of ChREBP in Fatty Acid Inhibition of Glucose Metabolism

Fatty acids inhibit transcription of L-PK and other enzymes in glycolysis and lipogenesis pathways, while excess glucose induces expression of these genes (Duplus, E., Glorian, M., and Forest, C. (2000) J. Biol. Chem. 275, 30749-30752). AMP-activated protein kinase (AMPK) is a protein kinase involved in lipid metabolism and shown to catalyze phosphorylation and inactivation of 3-hydroxy-3-methylglutaryl-CoA (HMG-CoA) reductase and acetyl-CoA-carboxylase, the rate limiting enzymes of cholesterol and fatty acid synthesis, respectively (Hardie, D. G. (1992) Biochim. Biophys. Acta 1123, 231-238; Hardie, D. G and Carling, D. (1997) Eur. J. Biochem. 246, 259-273).

This example demonstrates the role of fatty acid inhibition of ChREBP as a result of AMPK action.

A. Methods

Cloning of ChREBP Gene from Rat Liver

Results of mass spectrometry analysis of triptic peptides of the purified ChREBP, indicated that ChREBP matched with sequences in the mouse putative hepatic transcription factor, WBSCR14 (accession number AF156604) (de Luis, O., Valero, M. C., and Jurado, L. A. (2000) Eur. J. Hum. Genet. 8, 215-222), with variable degrees of identity. To obtain a portion of rat ChREBP gene, two degenerate primers were designed based on the sequences of human and mouse WBSCR14. The degenerate oligonucleotide primer sequences were forward primer, 5′-ATHCAYWSNGGNCAYTTYATGG-3′ (SEQ ID NO: 57) and reverse primer, 5′-GTNCCYTCNGTNACNGCNCKNG-3′ (SEQ ID NO: 58). Total RNA was extracted from rat liver by ISOGEN (Nippon Gene, Toyama, Japan). The first stranded cDNA was synthesized from poly (A)⁺ RNA prepared by mRNA Purification Kit (Amersham Pharmacia Biotech, Piscataway, N.J.) using SuperScript II reverse transcriptase (Life Technologies, Grand Island, N.Y.). The PCR reaction mixture, in a final volume of 50 μl, contained 10 mM Tris-HCl, pH 8.3, 50 mM KCl, 1.5 mM MgCl₂, 200 μM dNTPs, 1 μM of each primer, 5 μl of cDNA, and 5 U of AmpliTaq Gold DNA polymerase (Applied Biosystems, Foster, Calif.). The PCR product was purified from an agarose gel and cloned into a pGEM-T Easy vector (Promega, Madison, Wis.). The DNA sequence of the cloned fragment was confirmed using the dideoxy chain termination method, with the BigDye Terminator Cycle Sequencing Kit and an ABI 377 DNA sequencer (Applied Biosystems). Based on the sequence of this cDNA fragment obtained using the degenerate primers, two additional pairs of primers were designed and used in 5′- and 3′-rapid amplification of cDNA ends (5′- and 3′-RACE) in an effort to generate a cDNA containing the entire length of rat the ChREBP gene. In these reactions, it was necessary to use a nested primer to generate a fragment of the correct size. The antisense primer and its nested primer were 5′-CCCAAGCAGCACAGGCACCAC-3′ (SEQ ID NO: 59) and 5′-GCTCTTCCTCCGTTGCACATACTG-3′ (SEQ ID NO: 60) to generate the fragment of cDNA corresponding to the 5′-end. The sense primer and its nested primer used in 3′-RACE were 5′-ACCCGCACGCTGCACAACTGGAAG-3′ (SEQ ID NO: 61) and 5′-CTGAGGGATGAAATAGAGGAGCTC (SEQ ID NO: 62). The 5′-RACE product (450 bp) and 3′-RACE product (980 bp) were cloned into the pGEM-T Easy vector; the sequences of three independent clones of each were verified by DNA sequencing as described above.

Plasmids and Mutagenesis

The full-length rat wild type-ChREBP cDNA was ligated into the mammalian expression vector pcDNA3 (ChREBP/pcDNA3) (Invitrogen, Carlsbad, Calif.) (Yamashita et al. Proc. Natl. Acad. Sci. USA 98, 9116-9121) or pEGFP-N3 (ChREBP/pEGFP) (CLONTECH, Palo Alto, Calif.) encoding enhanced green fluorescent protein (GFP). The L-PK gene promoter region, between positions −206 and −7, was ligated into the luciferase expression plasmid, pGL-3 Basic vector (Promega, Madison, Wis.), as previously described (Yamashita et al. Proc. Natl. Acad. Sci. USA 98, 9116-9121). A truncated derivative of the C-terminus of ChREBP (amino acids 540-804) was synthesized with PCR and inserted at the Bam HI and Hind III sites of the T7-based expression vector, pGEX-5 (Amersham Pharmacia Biotech) to produce an N-terminal fusion with glutathione reductase. Point mutations in the ChREBP clones were created in the putative phosphorylation sites for AMPK using QuickChange Site-Directed Mutagenesis Kit (STRATAGENE, La Jolla, Calif.) according to the manufacturer's instruction.

Primary Hepatocyte Culture and Transfection

Primary hepatocytes were prepared as described in Example 3.

After a six h attachment period, hepatocytes were transfected using synthetic liposome (Lipofectamine 2000; Life Technologies) in Opti-MEM I reduced serum medium as described (Yamashita et al. Proc. Natl. Acad. Sci. USA 98, 9116-9121) and incubated for 12 h. As a control, hepatocytes were exposed to synthetic liposome in the same manner as each experimental group. The media containing the liposome-DNA complex was removed and replaced with glucose-free Dulbecco's modified Eagle's medium supplemented with 27.5 mM glucose plus either 150 μM albumin-bound specific fatty acids at a fatty acid/albumin ratio of 4:1 or 5-amino-4-imidazolecarboxamide ribotide (AICAR). The source of albumin for all studies was essentially fatty acid-free bovine serum albumin.

AMP, ADP, and ATP Assays

Hepatocytes were scraped and collected directly into 0.5 ml of ice-cold 6% (v/v) HClO₄. The concentrations of ADP and ATP were determined enzymatically in the acid-extract with methods described (Kawaguchi, T., Veech, R. L., and Uyeda, K. (2001) J. Biol. Chem. 276, 28554-28561), and the cytosolic AMP was calculated by the method of Veech et al. (Veech, R. L., Lawson, J. W., Cornell, N. W., and Krebs, H. A. (1979) J. Biol. Chem. 254, 6538-6547).

Measurement of AMPK Activity

Hepatocytes were directly lysed in the culture medium by adding 1.5 ml of buffer A (50 mM Tris-HCl, pH 7.5, 50 mM NaF, 5 mM sodium pyrophosphate, 1 mM EDTA, 10% glycerol, 1 mM dithiothreitol)+1% Triton X−100. The extract was centrifuged at 15,800·g for 15 min, and the resulting supernatant solution was transferred to a fresh tube, adjusted to 10% with polyethylene glycol 8000 (Appligene, Illkirich, France), and incubated on ice for 20 min. Following centrifugation at 15,800·g for 15 min, the pellet was suspended in 100 μl buffer A. Aliquots were used to assay the AMPK activity with the SAMS peptide (synthetic peptide HMRSAMSGLHLVKRR (SEQ ID NO: 63)) as a substrate and [γ-32P] ATP (specific activity, 3000 Ci/mmol) in the presence of 200 μM 5′-AMP as described previously (da Silva Xavier, G., Leclerc, I., Salt, I. P., Doiron, B., Hardie, D. G., Kahn, A., and Rutter, G. A. (2000) Proc. Natl. Acad. Sci. USA. 97, 4023-4028).

Determination of Luciferase Activity

Transfected cells were cultured for 12 h in culture medium containing 27.5 mM glucose plus 150 μM albumin-bound specific fatty acids or AICAR, washed twice with 2 ml Ca₂₊- and Mg₂₊-free phosphate-buffered saline, and lysed with Passive lysis buffer (Promega). Firefly luciferase and renilla (sea pansy) luciferase activities were measured sequentially using a Dual-Luciferase Reporter assay system (Promega) and a model TD-20E Luminometer (Turner Design, Sunnyvale, Calif.). After measuring the firefly luciferase signal and the renilla luciferase signal, the index of relative luciferase activity, indicating L-PK transcription activity, was calculated according to manufacturer's instructions (Promega).

B. Results

Identification and Cloning of Rat ChREBP Gene

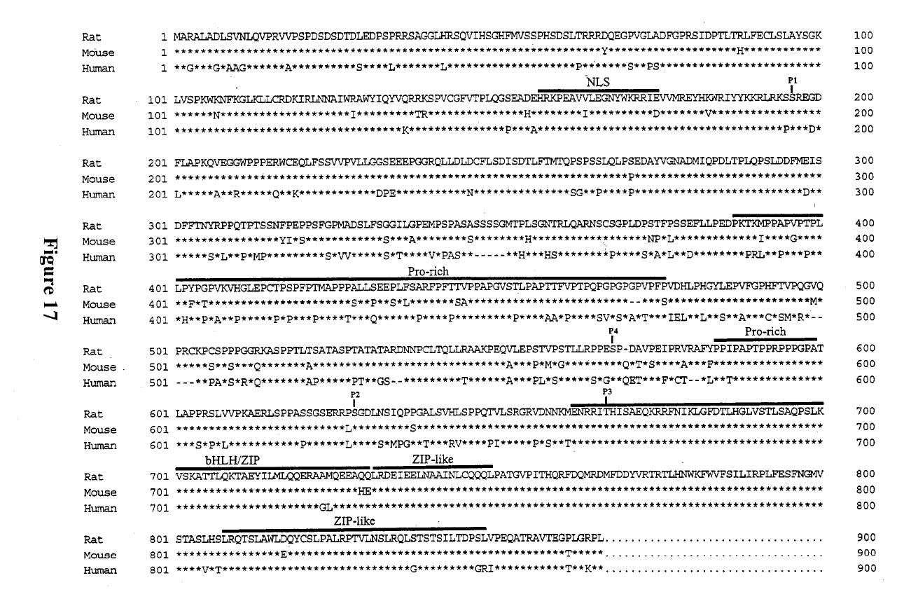

Rat liver cDNA fragment encoding ChREBP was generated by PCR using the degenerate oligonucleotide primers, and sequence of the cloned cDNA was determined. Based on the sequence of this fragment, additional gene-specific primers were synthesized and used in 5′- and 3′-RACE, which produced 450 and 980 bp cDNA fragments. The resulting sequences overlapped and allowed determination of the complete nucleotide sequence of the ChREBP cDNA. The rat ChREBP cDNA encodes an 865 amino acids protein (a predicted Mr=94,780). The rat ChREBP showed identities of 93.8% and 82.0% with amino acid sequences of the mouse and human counterparts, respectively, with the C-terminal region (amino acids 652-865) highly conserved (>95% identity) (FIG. 17). The alignment shows the location of functional domains, a nuclear localization signal (NLS) domain, a proline-rich stretch (Pro-rich) domain, a basic helix-loop-helix and leucine-zipper (bHLH/ZIP) domain, and a leucine zipper-like (ZIP-like) domain. Phosphorylation sites of cAMP-dependent protein kinase are indicated as P1, P2, and P3. The putative phosphorylation site of AMPK is P4. In contrast to the other species, the rat ChREBP had an additional PKA phosphorylation site at Ser⁵¹⁶.

Effects of Various Fatty Acids on Glucose-Induced L-PK Transcriptional Activation in ChREBP Overexpressed Hepatocytes.

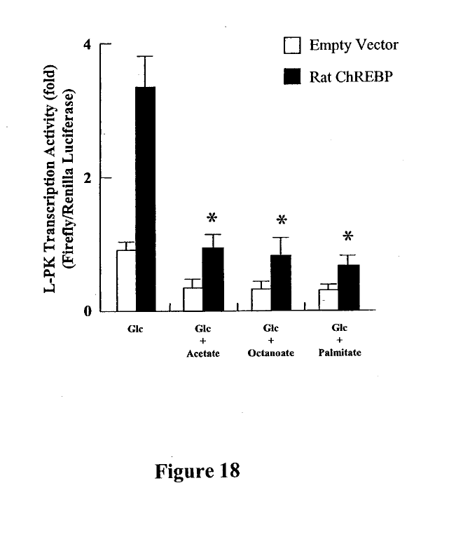

Hepatocytes transfected with ChREBP and maintained in medium containing high glucose (27.5 mM) expressed high L-PK transcriptional activity (FIG. 18). Addition of 150 μM acetate, octanoate, or palmitate to the high glucose medium inhibited the glucose activation by at least 75% (FIG. 18). A higher concentration (1 mM) of fatty acids eliminated the glucose activation completely. Thus, all these fatty acids inhibited equally the ChREBP-dependent glucose activation of L-PK transcription.

To determine if this inhibition of transcription was due to inhibition of the DNA binding activity of ChREBP, a gel shift assay of extracts of those hepatocytes was performed (1×10⁶ cells), but no DNA binding activity was detectable in any of the hepatocyte extracts including those grown in glucose alone, suggesting that the activity of ChREBP was too low to detect.

Effects of Various Fatty Acids on AMP/ATP Ratio and AMPK Activity in Hepatocytes

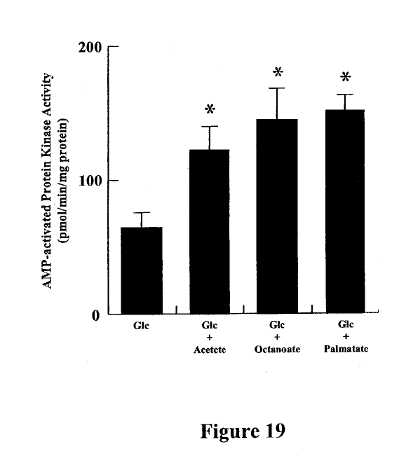

In the activation of fatty acids to fatty acyl-CoA, AMP is generated in the cytoplasm. Adenine nucleotide concentrations in perchloric acid extracts of hepatocytes incubated in the presence of fatty acids were determined. The cytosolic AMP increased approximately 30-fold, whereas ADP and ATP remained constant in the presence of fatty acids (Table 5). The 30-fold rise in AMP/ATP ratio is sufficient for activation of AMPK, which is known to play a central role in regulation of lipid metabolism. By addition of acetate, octanoate, or palmitate to the high glucose medium, AMPK activity also was increased about two-fold compared to those in the absence (FIG. 19). Thus, these results suggested that the fatty acids activated AMPK as a result of the increase in the AMP/ATP ratio in hepatocytes, and the fatty acid inhibition of the glucose-induced L-PK transcription resulted from the activation of AMPK which inactivated ChREBP.

Effect of AICAR on Glucose-induced Transcriptional Activation of the L-PK gene.

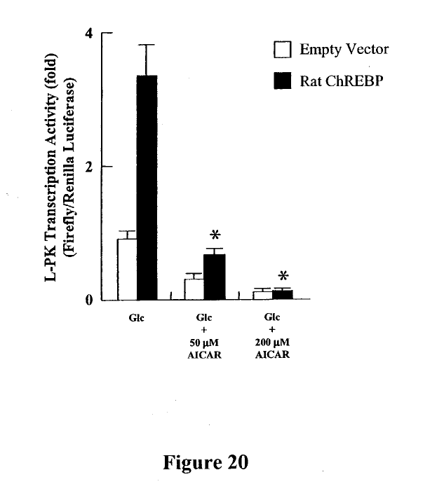

In order to confirm that the activated AMPK inhibits ChREBP-mediated transcriptional activation of the L-PK gene in cultured hepatocytes, the effect of AICAR was examined. AICAR is a specific activator of AMPK that mimics AMP in intact cells. AICAR at 50 μM and 200 μM inhibited the glucose activation of L-PK gene transcription approximately 80% and over 90%, respectively (FIG. 20), suggesting that the activated AMPK phosphorylated ChREBP, resulting in inhibition of the glucose activation of L-PK gene transcription.

TABLE 5
AMP, ADP, ATP concentrations, and AMP/ATP ratio
in cultured hepatocytes in the presence of fatty acids

| | AMP | ADP | ATP | |
|---|---|---|---|---|
| | (nmol/g cells) | (mol/g cells) | (mol/g cells) | AMP/ATP ratio |
| Glucose | 35 ± 6.1. | 28 + 0.08 | 3.11 ± 0.32 | 0.011 ± 0.0007 |
| Glucose + Acetate | 910 ± 87* | 1.32 ± 0.11 | 2.99 ± 0.36 | 0.30 ± 0.01* |
| Glucose + Octanoate | 942 ± 106* | 1.29 ± 0.15 | 3.09 ± 0.55 | 0.30 ± 0.03* |
| Glucose + Palmitate | 956 ± 95* | 1.31 ± 0.14 | 2.91 ± 0.41 | 0.33 ± 0.01* |

*p < 0.05 compared with glucose alone

Hepatocytes were incubated for 12 h with 150 M acetate, octanoate, or palmitate. Values are expressed as mean±S.E. (n=4).

### Example 17

### Characterization of Cytosolic Phosphatase of ChREBP

A. Methods

[γ-32P]ATP (3000 Ci/mmol) was purchased from Amersham Pharmacia Co. The PKA catalytic subunit was from New England Biolabs (Beverly, Mass.). AMPK was purchased from Upstate Biotechnology (Lake Placid, N.Y.). Okadaic acid and other inhibitors were from Calbiochem (San Diego, Calif.). DEAE-cellulose (DE52) was purchased from Whatman (Hillsboro, Oreg.), and Mono Q column from Amersham Biosciences (Piscataway, N.J.). All other chemicals were reagent grade and obtained from commercial sources.

Animals

Male Sprague-Dawley rats weighing 200-250 g were purchased from Sasco Co (Omaha, Nebr.). Rats were starved for 48 h and then fed 24 h with the National Institutes of Health high sucrose diet lab chow (by weight: 20% casein, 60% sucrose, 15% cellulose, 2.5% minerals, and 2.5% vitamins). All rat experiments were conducted in accordance with the National Institutes of Health Guidelines for the Care and Use of Laboratory Animals.

Preparation of Cytosolic Fraction of Liver Extract.

Liver was homogenized in a motor-driven Potter-Elvehjem homogenizer in 1 volume of extraction buffer containing 10 mM Hepes (pH 7.9), 10 mM KCl, 0.1 mM EDTA, 0.74 mM spermidine, 0.25 M sucrose, 50 mM imidazole, 1 mM dithiothreitol (DTT), 4 mM phenylmethanesulfonyl fluoride, and 1 mM benzamidine, and the extract was centrifuged at 10,000×g for 30 min.

Preparation of Nuclear Extracts.

Liver nuclear extracts were prepared as described in Example 1.

Preparation of Phosphorylated Substrates and Protein Phosphatase Assay.

Synthetic peptides corresponding to amino acids #187-201 (YYKKRLRKSSREGDF “P1” peptide) (SEQ ID NO:64), #556-573 (STVPSTLLRPPESPDAVP, “P4” peptide) (SEQ ID NO:65), and #655-674 (VDNNKMENRRITHISAEQKR; “P3” peptide) (SEQ ID NO:66), of ChREBP were prepared by the Peptide Synthesis Service of University of Texas Southwestern Medical Center. These peptides were phosphorylated by 10 U of PKA for 6 h at 30° C. P4 peptide corresponding to 556-573 (STVPSTLLRPPESPDAVP) (SEQ ID NO:65), was phosphorylated by 10 U of AMPK for 5 h at 30° C. The reaction mixtures were passed through on a Dowex AG1X8 column (0.5 cm×5 cm) which had been equilibrated with 30% acetic acid to remove residual [³²P]-ATP. The labeled peptides in the pass through fraction were lyophilized.

Protein phosphatase activity was determined by the method of Deana et al (Deana et al. (1990) Biochem, Biophys. Acta. 1051: 199-202). The reaction mixture contained in a final volume of 50 μ1,20 mM MOPS, pH 7.0, 1 mM DTT, 25 μg of BSA, and ³²P-labeled substrate for 15 min at 30° C. The reaction was terminated by the addition of 10% trichloroacetic acid. Phosphate released was converted to a phosphomolybdic complex followed by organic solvent (isobutyl alcohol:heptane, 1:1) extraction of the complex. The reaction mixture was mixed thoroughly and centrifuged. An aliquot of the top layer was then added to 5 ml scintillation fluid and counted. One unit of protein phosphatase is defined as that amount which catalyzes the release of 1.0 μmol of ³²P/min at 30° C.

Construction of Plasmids.

Full length rat ChREBP cDNA (accession number AB074517) was ligated into a mammalian expression vector pcDNA3 (Invitrogen, Carlsbad, Calif.) or a pEGFP-N3 vector encoding enhanced green fluorescent protein (GFP) (Clontech, Palo Alto, Calif.). The promoter region between positions −206 and −7 of the LPK gene was ligated into the luciferase expression plasmid, pGL-3 basic vector (Promega/Madison, Wis.), as previously described (Kawaguchi et al (2001) Pro. Natl. Acad. Sci. 98:13710).

Primary Hepatocyte Culture and Transfection.

Primary hepatocytes were prepared from male Sprague-Dawley rats (250-300 g) using the collagenase perfusion method (Yamashita et al. (2001) Pro. Natl. Acad. Sci. 98:9116-9121) and plated in collagen-coated 35 mm tissue culture plates (Primaria Falcon/Franklin Lakes, N.J.) at a density of 1.0×10⁶ cells/well in glucose-free Dulbecco's modified Eagle's medium (Life Technologies, Inc.) supplemented with 10 nM dexamethasone, 0.1 unit/ml insulin, 100 units/ml penicillin, 100 μg/ml streptomycin, 10% dialyzed fetal bovine serum, and 5.5 mM glucose. After a 6 h attachment period, hepatocytes were transfected using synthetic liposome (Lipofectamine 2000 from Life Technologies, Inc.) in Opti-MEM I reduced serum medium as described (14) and incubated for 12 hr. The media containing the liposome-DNA complex was removed and replaced with glucose free Dulbecco's modified Eagle's medium supplemented containing 27.5 mM glucose.

Determination of Subcellular Localization of ChREBP

Microscopy was perforemed as described in Example 9.

Relative Luciferase Activity Assay

Transfected cells were cultured for 12 hr after replacement with culture medium containing 27.5 mM glucose, washed twice with 2 ml of Ca²⁺ and Mg²⁺ free phosphate buffered saline, and lysed with Passive Lysis Buffer (Promega). Luciferase activity assay was performed as described previously (Kawaguchi et al (2001) Pro. Natl. Acad. Sci. 98:13710). After measuring the firefly luciferase signal (LAF) and the renilla (sea pansy) luciferase signal (LAR), the relative luciferase activity (RLA) was calculated as: RLA=LAF/LAR, where relative RLA was calculated as a percentage, i.e. % RLA=RLA/(RLA)max. To compare the relative luciferase activity in one cell line with another, an index of relative luciferase activity was calculated as IRLA=RLA/RLASV40, where RLASV40 is the ratio of firefly luciferase signal with an SV40 promoter in pGL3 divided by the renilla luciferase signal in pRL-TK.

Metabolite Measurements

The freeze-clamped livers were ground in a porcelain mortar prechilled with liquid nitrogen, and the powder was stored at −70° C. until analysis. HClO₄ extracts of frozen livers were prepared as described (Veech et al. (1972) Biochem J. 127:387-397). Xu-5-P was assayed by the method of Casazza and Veech except the formation of fructose-6-phosphate coupled to NADPH formation was measured fluorometrically by adding phosphoglucose isomerase and glucose-6-P-dehydrogenase with excitation and emission wavelengths at 354 and 452 nm, respectively. All other metabolites were assayed spectrophotometrically by enzymatic methods (Lowry et al. (1982) A Felxible System of Enzymatic Analysis, Academic Press, New York pp. 146-218).

B. Results

Dephosphorylation of P1 Site and Activation of Nuclear Translocation

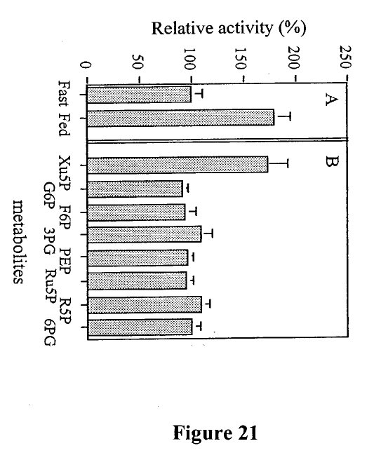

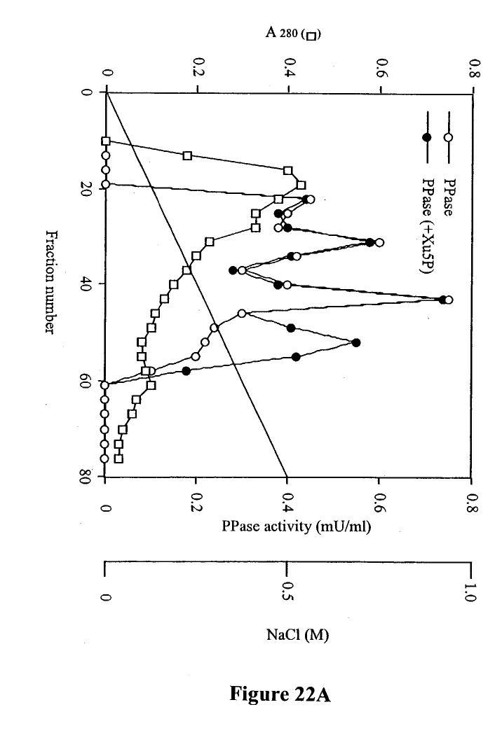

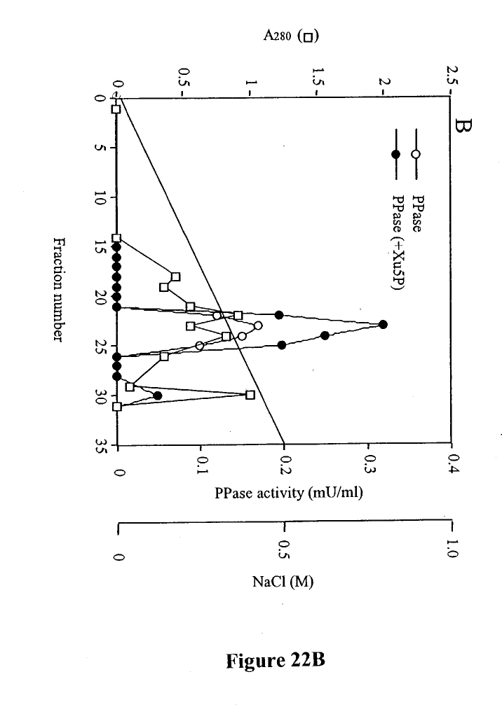

The first step in glucose activation of ChREBP is the dephosphorylation of Ser196 located near the NLS site. In order to investigate this reaction a synthetic peptide containing amino acids #187-201 of ChREBP was phosphorylated with a catalytic subunit of PKA and used as a substrate to search for a protein phosphatase (PPase) and a metabolite that activated the PPase. The source of the PPase and the metabolite was cytosolic fraction of liver extract of rats fed a high carbohydrate diet compared with a similar liver extract from fasted rats. The PPase activity in the high carbohydrate fed liver extract was about 1.8 times higher than that of the fasted liver extract (FIG. 21A, left panel).

In order to demonstrate the possible presence of a metabolite which may serve as an activator of the PPase, the extracts were desalted by passing through Sephadex G50 to remove endogenous metabolites. The effect of various intermediates of glycolysis and pentose shunt pathway was examined as a possible activator of the PPase. The results showed that among those glucose metabolites examined, only Xu-5-P was able to activate the dephosphorylation reaction catalyzed PPase reaction (FIG. 21B, right panel). The extent of the activation was close to that observed with the crude extract shown in the same figure, suggesting that Xu-5-P could account completely for the activation in the extract.

Characterization of the Cytosolic PPase

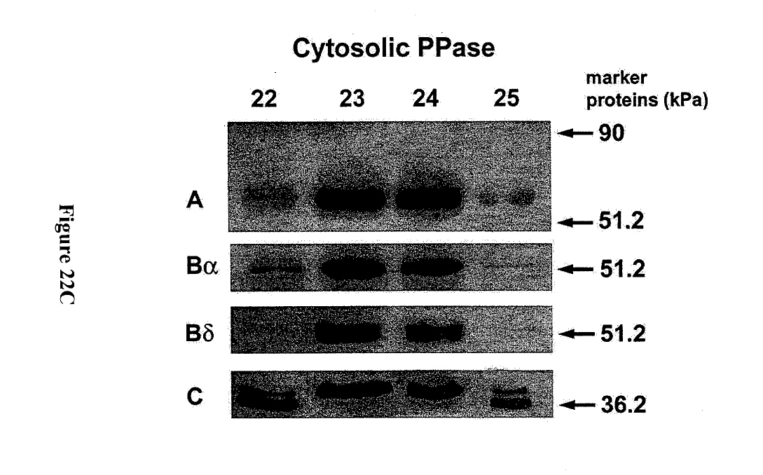

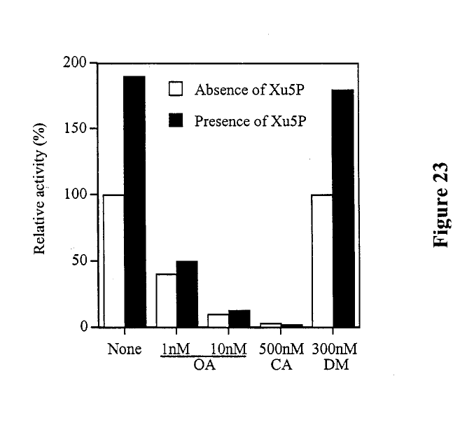

In order to characterize the cytosolic Xu-5-P activated PPase responsible for dephosphorylation of P1 site of ChREBP, the cytosolic extract was chromatographed on a DEAE-cellulose column as described previously (Veech et al. (1972) Biochem J. 127:387-397). PPase activity was assayed using P1 peptide as a substrate in the absence (open circle) and presence (black circle) of 50 μM Xu-5-P. Squares indicate absorbance, A₂₈₀. At least three peaks of PPase activity were detectable (FIG. 22), and these peaks appeared to correspond to the PPases reported by Pelech et al (24). However, none of these PPases was activated by Xu-5-P. The Xu-5-P-activated PPase was eluted at 0.35 M NaCl after the three major PPases had been eluted, and this PPase was only detectable in the presence of Xu-5-P (FIG. 22A).

To characterize the PPase further the fractions containing the Xu-5-P-activated PPase from the DEAE-cellulose chromatography (fractions #48-56) were pooled and further purified by chromatography on a Mono-Q column. Column was prepared with a linear gradient of 0-0.05M NaCl in 50 mM Tris-HCl, 0.1 mM EDTA, 1 mM DTT. A major and a minor peaks of PPase were eluted from the column (FIG. 22B). The major peak representing over 90% of the PPase activity was activated by Xu-5-P, but the minor peak, less than 10%, was insensitive to the pentose-P activation. Xu-5-P activated equally all the PPase in the eluted fractions, suggesting that the major peak contained one type of Xu-5-P-activated PPase free of any other PPases. Immunoblots of these fractions (fraction # 22-25) with antibodies against PP2A subunits A, Bα, Bδ, and C indicated that the Xu-5-P activated PPase was a trimer consisted of A, Bδ, C subunits (FIG. 22C). Furthermore, the Bδ peak as well as the A and the C peaks corresponded exactly with the Xu-5-P activated peak. The antibodies against Bδ is specific for this isoform, but the antibodies against Bα reacted with both Bα and Bδ. The immunoreactivity indicated that the regulatory subunit is likely Bδ, and the estimated molecular weights of the subunits were identical to those we reported previously (Nishimura et al. (1995) J. Bio. Chem. 270:26341). Furthermore, the relative composition of these subunits in these PPase fractions appear same, suggesting the presence of the same PPase in these fractions.

In order to determine the type of the PPase, various inhibitors of PPases (MonoQ eluate) were examined. Okadaic acid, a potent inhibitor of PP2A (IC50=1 nM) (25), inhibited approximately 65% and 50% in the absence and presence of Xu-5-P (FIG. 23). At higher concentration (10 nM) okadaic acid inhibited completely in the absence and presence of Xu-5-P. Cantharidic acid, “CA” (500 nM), an indiscriminate inhibitor at this concentration, inhibited the Xu-5-P activated PPase completely (FIG. 23). However, delatmethrin, “DM”, (300 nM), a PP2B inhibitor, did not affect the activity. These results as well as the similarity of this Xu-5-P activated PPase with the previously found liver PPase (Veech et al. (1992) Biochem J. 127:387) and the above immunoreactivity suggested strongly that this PPase was type 2A.

Dephosphorylation of P3 Site and Activation of the DNA Binding Activity of ChREBP in Nuclear Extract

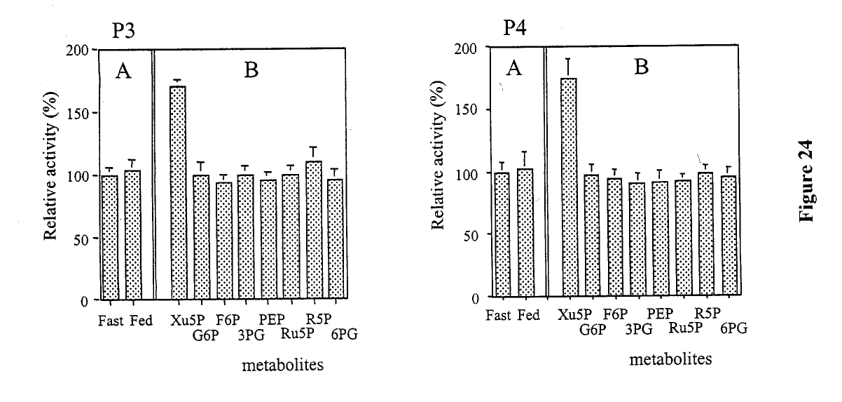

The DNA binding activity of ChREBP is inhibited by reversible phosphorylation of Thr666 (P3 site FIG. 24, left), which is located within a bHLH (Kawaguchi et al. (2001) Pro. Natl. Acad. Sci. 98:13710). The glucose activation of the DNA binding activity would be expected by dephosphorylation of the P3 site and should in nucleus. Thus, we searched for a specific PPase catalyzing the dephosphorylation reaction in the nucleus of liver of rats fed high carbohydrate. A synthetic peptide of the bHLH region corresponding to amino acids #655-674 containing the target Thr666 was prepared and phosphorylated by PKA and used as a substrate. As shown in FIG. 24A (left), PPase activity was present in the crude nuclear extracts from the fed rat liver, but, in contrast to the cytosolic extracts shown in FIG. 21A, the nuclear PPase activities in the nuclear extracts were the same in both the starved and fed rat livers. The nuclear PPase was activated by added Xu-5-P, which suggested that the metabolites were absent in the nuclear extract FIG. 24B (left). Similar to the cytosolic PPase, Xu-5-P was the only metabolite able to activate the nuclear PPase. The absence of the metabolites in the nuclear extract was probably due to loss of metabolites by diffusion during the isolation of the nuclei by gradient centrifugation. The lack of difference between the nuclear extracts of fed and fasted livers and full recovery of the enzyme activity with addition of Xu-5-P supported the idea that this pentose-P was the only factor necessary for the full activation of the PPase, and no other activator appeared to be required.

Dephosphoryaltion of P4 Site and Activation of the DNA Binding Activity

Dephosphorylation of P4 site using a peptide corresponding to the amino acids #556-573 ChREBP was catalyzed by nuclear PPase, which also was activated only by Xu-5-P (FIG. 24 right, B).

Characterization of the Nuclear Xu-5-P Activated PPases

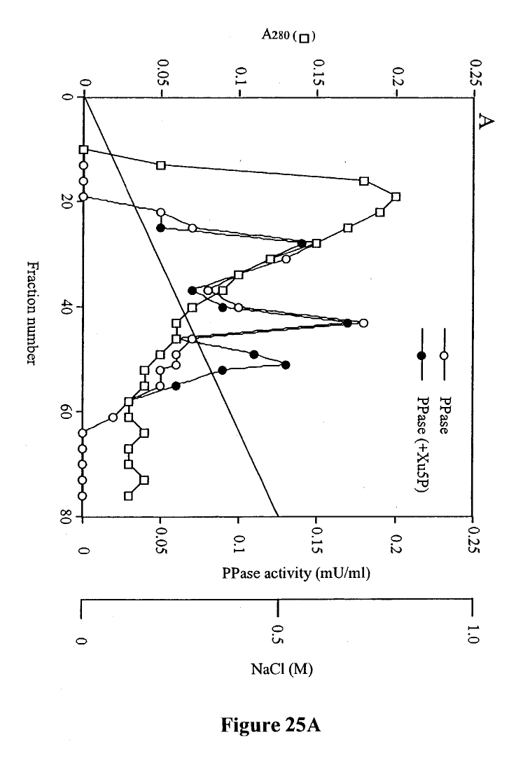

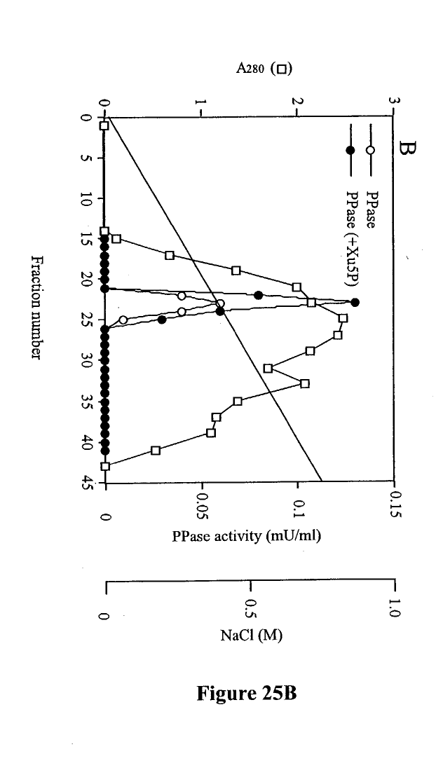

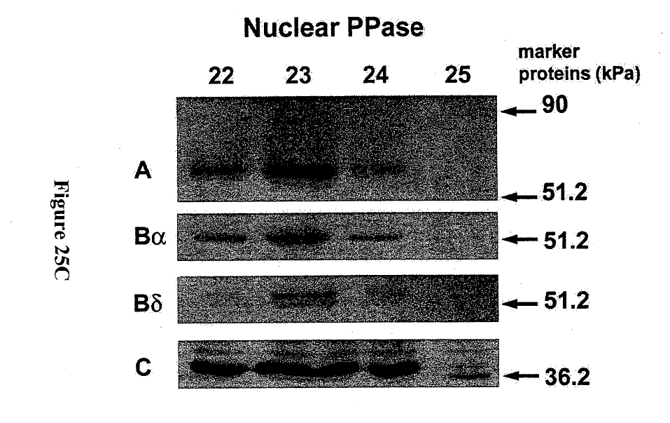

In order to characterize the nuclear Xu-5-P activated PPase, crude nuclear extract was chromatographed on DEAE-cellulose as shown in FIG. 25A. The elution pattern was identical to that of the cytosolic PPases, and the nuclear Xu-5-P-activated PPase was eluted at the same concentration (0.35 M) of NaCl as the cytosolic Xu-5-P activated PPase. The elution pattern of the nuclear PPase from the Mono Q column was also identical to that of the cytosolic PPase (FIG. 25B).

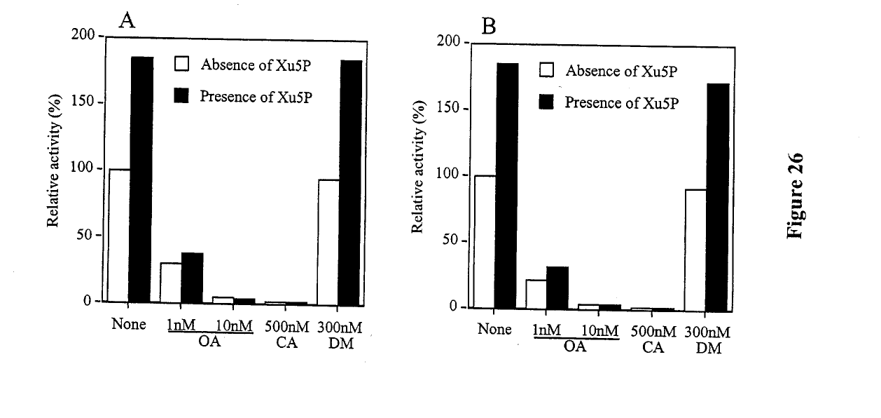

Xu-5-P-activated PPase in the nuclear extract (the Mono Q eluate) was inhibited by 1 nM okadaic acid 70% and 63% respectively, in the presence and absence of Xu-5-P, and over 95% by 10 nM okadaic acid. The nuclear PPase was also completely inhibited by 500 nM Canthradic acid, but was not affected by 300 nM deltamethrin (FIG. 26A). The same results were obtained with the PPase acting on the P4 peptide as the substrate (FIG. 26B).

Substrate specificities of the cytosolic and the nuclear Xu-5-P-activated PPases toward P1, P3 and P4 peptides were compared using the Mono Q purified enzymes, and the results are summarized in Table 6 The results indicated that both PPases were able to dephosphorylate all these site peptides, and showed essentially the same activity with respect to P1 and P2 peptides but somewhat lower activity with P3. Furthermore both enzymes were activated by Xu-5-P to the same extent (1.8 fold) (Table 6). All these data suggested that the cytosolic and nuclear PPases activated by Xu-5-P were similar PPases, if not identical. This conclusion was further supported by the results of immunoreactivity examined using the specific antibodies against PP2A-Bα and PP2A-Bδ. Both the cytoslic and the nuclear Xu-5-P activated PPase demonstrated immunoreactivity with Bα and more specific Bδ and suggest that these PP2A could be PP2ABδC isoform.

TABLE 6
A comparison of Xu-5-P activation of cytosolic and nuclear
PPase with different substrates.

| | Cytosol. PPase | | Nuclear PPase | |
|---|---|---|---|---|
| | (units/mg) | | (units/mg) | |
| Substrate | None | +Xu5P | None | +Xu5P |
| P1 peptide | 6.2 | 11.2 | 0.55 | 0.93 |
| P3 peptide | 6.0 | 11.0 | 0.52 | 0.94 |
| P4 peptide | 5.2 | 8.8 | 0.39 | 0.67 |

These peptides were assayed with Mono Q purified cytosolic and nuclear PPases in the presence and absence of 50 μM Xu-5-P.

Glucose-induced Kinetic Changes in Xu-5-P Level, ChREBP Translocation and Transcription Activity in Hepatocytes.

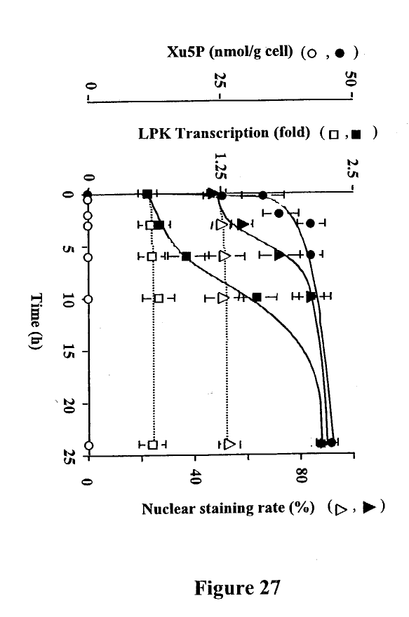

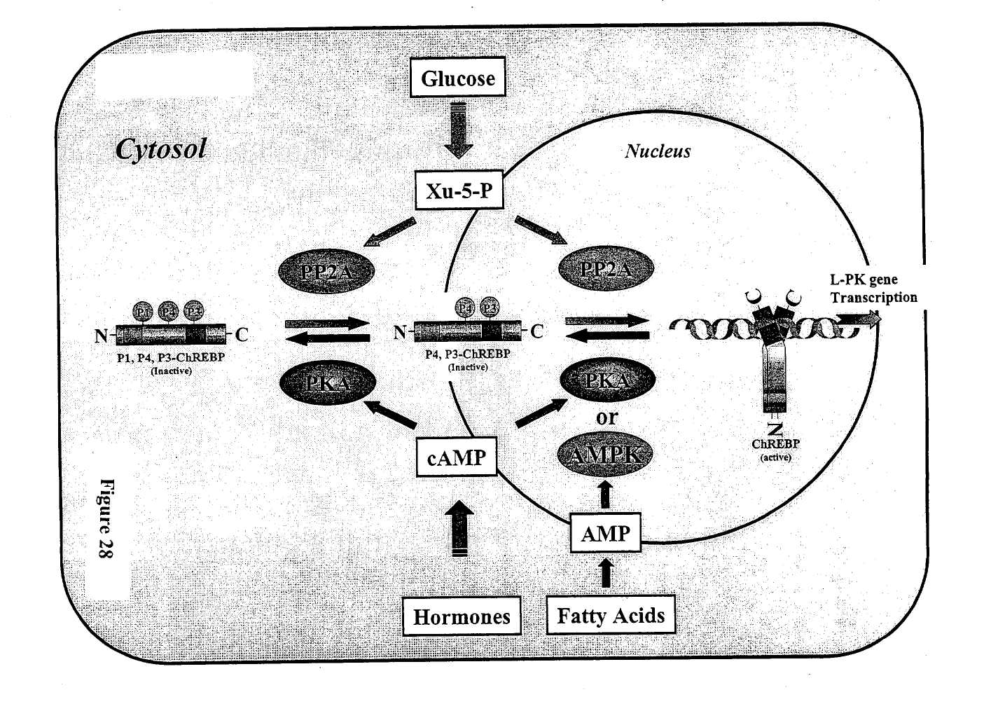

Glucose activation of LPK transcription requires ChREBP to be translocated from cytosol to nucleus by dephosphorylation of the P1 site near the NLS, followed by activation of the DNA binding activity by dephosphorylation of the P3 and/or P4 sites near or within the bHLH domain. If Xu-5-P is indeed the glucose signaling compound essential for the activation of ChREBP and LPK transcription, then the formation of Xu-5-P must precede the nuclear import which is followed by transcription. The kinetics of these three events in primary hepatocytes transfected with GFP-fused ChREBP or ChREBP and incubated in high (closed symbols, 27.5 mM) and low glucose. As shown in FIG. 27, the formation of Xu-5-P was quite rapid and took less than 30 min to reach 80% of the maximum and it remained at the maximum concentration of 43 mmol/g cell for 24 h under high glucose. The nuclear translocation began in approximately 3 h and reached half maximum at about 5 h and maximum at 10 h. The LPK transcription also commenced slowly after 3 h, reached a half maximum in about 10 h and the maximum after approximately 15 h. However, in low glucose medium, the Xu-5-P formation, ChREBP translocation and the LPK transcription remained constant (FIG. 27). These results are consistent with the proposed sequence of reactions in the glucose-stimulated pathway of ChREBP activation.

All references cited herein are incorporated herein in their entirety.

I claim:

1. A method for modulating expression of a DNA molecule that encodes a protein involved in glucose metabolism or lipogenesis in a cell, wherein said DNA expression is regulated by ChREBP (carbohydrate response element binding protein), comprising
contacting the cell with an agent that induces phosphorylation or dephosphorylation of ChREBP,
wherein dephosphorylated ChREBP induces expression of the DNA molecule.

2. The method of claim 1 wherein the dephosphorylated ChREBP is transported to the nucleus of the cell and binds to the DNA molecule.

3. A method of inhibiting lipogenesis or glycolysis in a cell, wherein expression of a DNA molecule that encodes a protein involved in glucose metabolism or lipogenesis is suppressed, wherein said DNA expression is regulated by ChREBP, comprising
contacting the cell with an agent that modulates phosphorylation of ChREBP,
wherein dephosphorylated ChREBP induces expression of the DNA molecule and phosphorylated ChREBP does not induce expression of the DNA molecule.

4. A method of treating obesity, diabetes, or vascular diseases in an individual, wherein expression of a DNA molecule that encodes a protein involved in glucose metabolism or lipogenesis is suppressed in a cell such that the protein is not produced, said DNA expression being regulated by ChREBP comprising
administering to the individual an agent that induces phosphorylation of ChREBP,
wherein dephosphorylated ChREBP induces expression of the DNA molecule and the protein involved in glucose metabolism or lipogenesis is produced.

5. The method of claim 1 wherein the protein encoded by the DNA molecule is an enzyme or a hormone.

6. A method of identifying an agent that inhibits ChREBP binding to an oligonucleotide, said oligonucleotide derived from a regulatory response element of the promoter region of a DNA molecule encoding a protein involved in glucose metabolism or lipogenesis in a cell, comprising
contacting the cell with the agent,
measuring the effect of the agent on ChREBP binding, and
comparing ChREBP binding in the presence and absence of the agent
wherein dephosphorylated ChREBP binds to said oligonucleotide and phosphorylated ChREBP does not bind to said oligonucleotide.

7. The method of claim 6 wherein ChREBP binding is measured by expression of an indicator gene, wherein the indicator gene is operably linked to the regulatory response element of the promoter region.

8. The method of claim 7, wherein the indicator gene is luciferase.

9. A method of identifying an agent that inhibits ChREBP localization to a cell nucleus, wherein nuclear localization of ChREBP regulates expression of a DNA molecule that encodes a protein involved in glucose metabolism or lipogenesis, comprising
contacting the cell with the agent,
measuring the effect of the agent on ChREBP localization, and
comparing ChREBP localization in the presence and absence of the agent,
wherein dephosphorylated ChREBP localizes to the nucleus and phosphorylated ChREBP does not localize to the nucleus.

10. The method of claim 9, wherein ChREBP comprises a nuclear localization signal (NLS), wherein the candidate agent modulates the activity of the NLS, wherein blockage of NLS inhibits ChREBP localization to the nucleus.

11. The method of claim 1, 3, 4, 6, or 9, wherein the cell is a human cell.

12. The method of claim 1, 3, 4, 6, or 9, wherein the cell is a liver cell.

13. The method of claim 1, 3, 4, 6, or 9, wherein the agent is selected from the group consisting of phosphatase inhibitors and PP2A inhibitors.

15. The method of claim 1, 3, 4, 6, or 9, wherein the DNA molecule encodes a protein selected from the group consisting of L-type pyruvate kinase, fatty acid synthase, acetyl CoA carboxylase, insulin, and ATP citrate lyase.

16. A method of modulating carbohydrate metabolism in an individual, wherein expression of a DNA molecule that encodes proteins involved in glucose metabolism or lipogenesis is suppressed, comprising
administering to the individual an agent that induces phosphorylation of ChREBP,
wherein the phosphorylated ChREBP does not induce expression of the DNA molecule.

17. A method of modulating carbohydrate metabolism in an individual, wherein expression of a DNA molecule that encodes proteins involved in glucose metabolism or lipogenesis is suppressed, comprising
administering to the individual an agent that inhibits dephosphorylation of ChREBP,
wherein the phosphorylated ChREBP does not induce expression of the DNA molecule.

18. The method claim 16 or 17 wherein the subject has obesity, diabetes or a vascular disorder.
